SEQUENCE LISTING (20 sequences)

SEQ ID NO: 1 - DNA, 21 bp, Artificial sequence
  a sense strand of siRNA against TRPV1
gcaucuucuacuucaacuunn

SEQ ID NO: 2 - DNA, 21 bp, Artificial sequence
  a sense strand of siRNA against TRPV1 (Christoph et al., 2006)
gcgcaucuucuacuucaactt

SEQ ID NO: 3 - DNA, 21 bp, Artificial sequence
  a sense strand of siRNA against NOX3
gguggugagucacccaucunn

SEQ ID NO: 4 - DNA, 21 bp, Artificial sequence
  an antisense strand of siRNA gainst TRPV1
aaguugaaguagaagaugcnm

SEQ ID NO: 5 - DNA, 21 bp, Artificial sequence
  an antisense strand of siRNA against TRPV1 (Christoph et al., 200 6)
guugaaguagaaguagcgctt

SEQ ID NO: 6 - DNA, 21 bp, Artificial sequence
  an antisense strand of siRNA against NOX3
agaugggugacucaccaccnn

SEQ ID NO: 7 - DNA, 21 bp, Homo sapiens
gcgcatcttctacttcaactt

SEQ ID NO: 8 - DNA, 21 bp, Homo sapiens
aaggtggtgagtcacccatct

SEQ ID NO: 9 - DNA, 20 bp, Artificial sequence
  NOX3 sense primer
gtgaacaagggaaggctcat

SEQ ID NO: 10 - DNA, 19 bp, Artificial sequence
  NOX3 antisense primer
gacccacagaagaacacgc

SEQ ID NO: 11 - DNA, 21 bp, Artificial sequence
  rodent GAPDH sense primer
atggtgaaggtcggtgtgaac

SEQ ID NO: 12 - DNA, 21 bp, Artificial sequence
  rodent GAPDH antisense primer
tgtagttgaggtcaatgaagg

SEQ ID NO: 13 - DNA, 20 bp, Artificial sequence
  rodent TRPV1 sense primer
caaggctgtcttcatcatcc

SEQ ID NO: 14 - DNA, 20 bp, Artificial sequence
  rodent TRPV1 antisense primer
agtccagtttacctcgtcca

SEQ ID NO: 15 - DNA, 20 bp, Artificial sequence
  rodent Rac-1 sense primer
atcagttacacgaccaatgc

SEQ ID NO: 16 - DNA, 20 bp, Artificial sequence
  rodent Rac-1 antisense primer
gggaaaagcaaattaagaac

SEQ ID NO: 17 - DNA, 21 bp, Artificial sequence
  rodent gp-91 sense primer
taaaggagtgcccagtaccaa

SEQ ID NO: 18 - DNA, 21 bp, Artificial sequence
  rodent gp-91 antisense primer
aatcccttcttcttcatctga

SEQ ID NO: 19 - DNA, 21 bp, Artificial sequence
  Rat p22 sense primer
acagggggcatcgtggctact

SEQ ID NO: 20 - DNA, 21 bp, Artificial sequence
  rat p22 antisense primer
ggacgtagtaatttctggtga

(19) United States
(12) Patent Application Publication
(10) Pub. No.: US 2011/0160279 A1
(43) Pub. Date: Jun. 30, 2011

(54) METHODS FOR TREATMENT AND PREVENTION OF OTOTOXICITY BY siRNA

(75) Inventors: Vickram Ramkumar, Springfield, IL (US); Debashree Mukherjea, Springfield, IL (US); Len Rybak, Springfield, IL (US)

(73) Assignee: BOARD OF TRUSTEES OF SOUTHERN ILLINOIS UNIVERSITY, Springfield, IL (US)

(21) Appl. No.: 12/673,419

(22) PCT Filed: Aug. 13, 2007
(86) PCT No.: PCT/US07/75826

§ 371 Date: Feb. 22, 2011

(51) Int. Cl. A61K 31/713; A61P 27/16

(52) U.S. Cl. 514/44 A

(57) ABSTRACT

The present invention relates to methods for reducing and/or preventing ototoxicity caused by an ototoxic agent, noise or head and/or neck radiation. It is also directed to a method for preventing or reducing generation of reactive oxygen species in the inner ear of a patient. The methods of the present invention include administering at least one siRNA directed against TRPV1 mRNA, NOX3 mRNA or a combination thereof.

### FIELD OF THE INVENTION

The present invention is directed to a method for preventing and/or reducing ototoxicity in a patient caused by an ototoxic agent, noise or head and neck radiation, by silencing a TRPV1 gene in the ear of a patient, e.g., by using a siRNA directed against TRPV1, administering a siRNA directed against NOX3, or a combination thereof. The present invention also relates to a method for preventing or reducing generation of reactive oxygen species in the inner ear of a patient by administering to the patient at least one siRNA directed against TRPV1 mRNA and/or NOX3 mRNA.

### BACKGROUND OF THE INVENTION

There are three major causes of hearing loss: noise-dependent hearing loss, drug-associated hearing loss and age-associated hearing loss. Interestingly, there appears to be a common mechanism to all three major causes of hearing loss, namely destruction of sensory epithelium and cochlear neurons through reactive oxygen species. In terms of treatment, no efficient drug treatment or prophylaxis of hearing loss are available at this point and the only option at present is the use of hearing aids. Tinnitus, also referred to as phantom hearing, is a common and in some instances invalidating medical complaint. Presently, the pathophysiology of the disease is poorly understood and there is no proven causative treatment available. There is however evidence that reactive oxygen species might play a role in the pathophysiology of tinnitus.

With respect to the drug-associated hearing loss, prolonged exposure of the cochlear cells to aminoglycosides is linked to the killing of outer hair cells in the organ of Corti and type I sensory hair cells in the vestibular organ, leading to permanent hair loss and vestibular damage. Damage to the hair cells progresses from the base of the cochlea (an area for high frequency sound detection) to the apex (an area for low frequency sound detection). This is followed by retrograde damage to the auditory nerve. The degree of hair cell damage and hearing loss is directly proportional to the dose of the drug to which the hair cells are exposed. Repeated exposure to aminoglycosides leads to an additive damage to hair cells and other structures and subsequently to deafness. Damage is more significant in the elderly who may have less hair cell or lower endogenous protective mechanisms or in other individuals with compromised auditory function. In addition, damage is generally potentiated by the concurrent administration of diuretics, such as ethacrynic acid and furosemide, which produce reversible hearing loss by themselves.

Furthermore, platinum containing drugs also cause ototoxicity and thereby hearing loss. Several reports have concluded that the generation of reactive oxygen species (ROS) is linked to cisplatin ototoxicity (reviewed by Forge and Schacht, 2000). Hearing loss due to cisplatin is usually permanent and cumulative. TRPV1 is a member of the transient receptor potential (TRP) channel family, expressed primarily by small diameter neurons (Aδ and C fibers) comprising the pain pathway. It is a nonselective cation channel which demonstrates responsivity to heat (Caterina et al., Nature 389, pp. 816-824, 1997). TRPV1 receptor expression has also been demonstrated in non-neuronal tissues including organ of Corti, keratinocytes and bladder urothelium (Zheng J et al. J. Neurophysiol. 90, 444-455, 2003; Southall et al., J. Pharmacol. Exp. Ther., 90, 444-455, 2003), suggesting additional roles in addition to the regulation of thermal pain sensation. The organ of Corti represents a major site for cisplatin induced hearing loss, a common side effect of this antineoplastic agent (Rybak, 1999). The mechanism underlying the hearing loss appears to involve the generation of reactive oxygen species (ROS) by this agent and permanent loss of outer hair cells (Kopke et al., Am. J. Otol., 18, 559-571, 1997). Therefore, new or improved methods are needed for alleviating ototoxicity.

In recent years, RNA interference (“RNAi”) has exhibited potential for use in many therapeutic applications. It refers to the process of sequence-specific post-transcriptional gene silencing in animals mediated by short interfering RNAs (siRNAs) (Fire et al., 1998, Nature, 391, 806).

The process of RNAi begins by the presence of a long dsRNA in a cell, wherein the dsRNA comprises a sense RNA having a sequence homologous to the target gene mRNA and antisense RNA having a sequence complementary to the sense RNA. The presence of dsRNA stimulates the activity of a ribonuclease III enzyme referred to as Dicer. Dicer is involved in the processing of the dsRNA into short pieces of dsRNA known as short interfering RNAs (siRNAs) (Berstein et al., 2001, Nature, 409, 363). Short interfering RNAs derived from Dicer activity are typically about 21 to about 23 nucleotides in length and comprise about 19 base pair duplexes (Elbashir et al., 2001, Genes Dev., 15, 188). siRNAs in turn stimulate the RNA-induced silencing complex (RISC) by incorporating one strand of siRNA into the RISC and directing the degradation of the homologous mRNA target.

The original RNAi, which was discovered in invertebrates and employed dsRNAs with length greater than 30 nucleotides was not effective in mammalian cells. This was found to be due to the fact that long dsRNAs (greater than 30 nucleotides) elicit interferon responses, resulting in nonspecific mRNA degradation and inhibition of protein synthesis. This problem was overcome by the finding that smaller double-stranded siRNAs with the length of 20-23 nucleotides do not induce an interferon response yet remain potent and specific inhibitors of endogenous gene expression (Elbashir et al., Nature 411, 494-498, 2001).

In research laboratories, two types of siRNA have been widely used to suppress exogenous as well as endogenous gene expression: synthetic siRNA and vector-based siRNA (i.e. in vivo transcribed siRNA). The vector-based siRNA is usually generated through short hairpin RNA (shRNA). In this system, RNA polymerase III promoters, such as H1 promoter and U6 promoter are used to drive transcription of shRNA. The shRNA transcript consists of a 19- to 29-bp RNA stem, with the two strands joined by a tightly structured loop. shRNA is processed in the cell into siRNA through the action of the Dicer family of enzymes. Thus, the transcribed products mimic the synthetic siRNA duplexes and are as effective as the synthetic siRNA for suppressing their corresponding genes.

### SUMMARY OF THE INVENTION

It is one embodiment of the present invention to provide a method for preventing and/or reducing ototoxicity in a patient suffering from or at risk from developing ototoxicity caused by an ototoxic agent, noise or head and/or neck radiation or tinnitus, wherein the method comprises silencing TRPV1 in a patient.

In another embodiment, the present invention relates to a method for preventing and/or reducing ototoxicity in a patient suffering from or at risk from developing ototoxicity caused by an ototoxic agent, noise or head and/or neck radiation or tinnitus, wherein the method comprises administering a siRNA directed against TRPV1 mRNA to the patient. In some alternative embodiments, a siRNA directed against NOX3 mRNA or a combination of siRNAs directed against TRPV- and NOX3 can be administered. In one preferred embodiment, a sense strand of the siRNA directed against TRPV1 has a sequence of SEQ ID NO: 1. In another preferred embodiment, a sense strand of the siRNA against TRPV1 has a sequence of SEQ ID NO: 2. In still another preferred embodiment, a sense strand of the siRNA directed against NOX3 has a sequence of SEQ ID NO: 3. In yet another preferred embodiment, the siRNA of the present invention is administered locally.

It is yet another embodiment of the present invention to provide a method for preventing or reducing generation of reactive oxygen species in inner ear of a patient, the method comprising administering to the patient at least one siRNA selected from siRNA directed against TRPV1 mRNA and NOX3 mRNA.

Other features of the present invention will be in part apparent and in part pointed out hereinafter.

### BRIEF DESCRIPTION OF THE DRAWINGS

FIG. 1 depicts the increased TRPV1 expression in organ of Corti on cisplatin treatment. (A) Cochleae from rats treated were harvested after 3 days post cisplatin treatment, sectioned and stained with TRPV1 antibody. Increased immunoreactivity was observed in cisplatin treated samples, compared to control as seen in the outer hair cells (OHC) and spiral ganglion (SG). (B) Increased TRPV1 protein expression was seen in the rat cochlea 72 hrs post cisplatin treatment. (C) TRPV1 mRNA expression in the rat cochlea increased as early as 24 hrs post cisplatin treatment, with no further significant change in expression levels over 48 and 72 hrs. (D) Lipoic acid pretreatment before cisplatin treatment (72 hrs) inhibited the upregulation of TRPV1 mRNA in rat cochlea suggesting involvement of ROS in the regulation of TRPV1. (E) Increased NOX3 mRNA expression was seen 72 hrs post cisplatin treatment in the rat cochlea. (F) Increased NOX3 mRNA expression was seen as early as 24 hrs post cisplatin treatment in the rat cochlea, with little or no further change at 48 and 72 hrs. (G) Lipoic acid pretreatment abolished the increase in NOX3 mRNA on cisplatin treatment (72 hrs), implicating ROS as the trigger for NOX3 induction in the cochlea. (*p<0.05 compared to control, students T test).

FIG. 2 shows increased TRPV1, NOX3, and other NADPH oxidase isoform expression following cisplatin administration in vitro. (A) Increased TRPV1 protein immunoreactivity was seen in UB/OC-1 cells treated with 2.5 μM cisplatin within 24 hrs post treatment. NADPH oxidase inhibitors like DPI and AEBSF downregulated the increase in TRPV1 immunoflourescence on cisplatin treatment 2.5 μM in UB/OC-1 cells implicating ROS in cisplatin mediated ototoxicity. (B) Graphical representation of average intensity of immunofluorescence seen in FIG. 2(A) is shown. (*p<0.05 compared to control, **p<0.05 compared to cisplatin, students T test). (C) Cisplatin 2.5 μM treatment increased TRPV1 protein expression significantly over control in UB/OC-1 cells. (D) Graphical representation of TRPV1 protein expression as shown in FIG. 2(C). (E) Three fold increase in TRPV1 mRNA expression was also observed in the UB/OC-1 cells on cisplatin 2.5 μM treatment (24 hrs) by real time RT-PCR. (F) UB/OC-1 cells on 30 minute cisplatin 2.5 μM treatment showed a marked increase in ROS generation as determined by DCF2DA dye. This increase in ROS generation was abolished by pretreatment with AEBSF an NADPH oxidase inhibitor, indicating a role for NADPH oxidases. (G) Increase in NOX3, gp-91 and Rac-1 mRNA was seen in UB/OC-1 cells treated with cisplatin (2.5 μM) that was abolished by pretreatment with DPI, an NADPH oxidase inhibitor. (H) Increased cell death was seen on 24 hr cisplatin treatment (20 μM) as seen by TUNEL. (I) Pretreatment with capsazepine and ruthenium red, antagonists of TRPV1 reversed the trend seen in FIG. 2(H) significantly. (*p<0.05 compared to control, **p<0.05 compared to control, student's T test). (J and K) Increased expression of pro-apoptotic proteins like p53 and Bax was seen in UB/OC-1 cells exposed to 24 hr cisplatin treatment over control cells. Pre-treatment with TRPV1 antagonist like capsazepine and calcium chelator like BAPTA-AM before cisplatin administration down-regulated the expression of these pro-apoptotic genes to control levels.

FIG. 3 depicts protective effects of TRPV1 siRNA directed against cisplatin induced ototoxicity in rat model. (A) UB/OC-1 cells were transfected with siNOX3 and siTRPV1 for 24 hrs and then treated with cisplatin 2.5 μM for another 24 hrs. NOX3 mRNA expression was reduced under basal levels as well as in cisplatin treated samples transfected with siNOX3. siTRPV1 did not change basal expression of NOX3 mRNA but did abolish the increase in NOX3 mRNA on subsequent cisplatin treatment. TRPV1 mRNA expression showed a similar pattern, suggesting co-regulation of these proteins in cisplatin induced ototoxicity. (B) Pretreatment ABRs were measured in rat models, followed by round window application of siTRPV1 (0.9 μg per ear) for 48 hrs, cisplatin administration (13 mg/kg i.p.) for 72 hrs and post ABRs were then collected. A significant decrease in threshold shift was observed at both 8 and 16 KHz in the siTRPV1 treated animals compared to the vehicle treated control animals implying otoprotection with siTRPV1. (C) TRPV1 mRNA expression in siTRPV1 treated cochlear samples showed an 85% down regulation compared to control cochlea. (D) Graphic representation of TRPV1 protein levels in the cochlea harvested from the siTRPV1 treated animals showed significant down regulation of TRPV1 compared to control. Pretreatment with siTRPV1 prior to cisplatin administration also showed significant decrease in TRPV1 expression compared to cisplatin treatment alone. (*p<0.05 compared to control, **p<0.05 compared to cisplatin, students T test). (E) Western blot of TRPV1 expression as described in FIG. 3(D). (F) Scanning electron microscopy images of the hook, basal turn and the middle turn of the cochleae of rats pretreated with either PBS or siTRPV1 48 hrs prior to cisplatin administration (72 hrs) showed significant protection in hair cell damage in all the turns. (G) shows the semi-quantitative graphical analysis of the percentage of hair cell death in all the samples from FIG. 3(F). Significant hair cell protection is seen in siTRPV1 treated cochleae compared to PBS treated controls at all the turns. (*p0.05 compared to PBS treated control, student's t-test). (H) is a diagram of a proposed mechanism for cisplatin-induced hair cell apoptosis.

FIG. 4 shows that noise exposure increases the expression of TRPV1 and NADPH oxidase subunits in the rat cochlea. (A) Rats were exposed to ambient noise (˜60 dB) (shaded bars) or 90 dB noise (open bars) for 8 h and auditory brainstem evoked responses (ABRs) measures were performed using test frequencies of 8 and 16 kHz. The 90 dB noise exposure induced a transient shift in ABR thresholds by ˜40 dB, compared to the pre-noise ABR (*, statistically significant difference, p<0.05). The 60 dB ambient noise levels did not produce significant shifts in ABR thresholds. (B) Demonstration of NAD(P)H activity in cochlear lysates using NADH and NADPH as substrates. (C) Noise increased NADPH oxidase activity in the rat cochlea at 1 and 3 days. (D) Noise exposure resulted in the induction of NADPH oxidase subunits, NOX3 and Rac1, along with TRPV1.

FIG. 5 depicts that inhibition of TRPV1 reduces generation of reactive oxygen species (ROS) by aminoglycosides. UB/OC-1 cultures were pretreated with vehicle (control), capsazepine (10 μM) or ruthenium red (20 μM) for 15 min prior to the administration of vehicle (control) or gentamicin (20 μM) for 30 min and the generation of reactive oxygen species was determined by the fluorescence of 2′,7′-dichlorodihydrofluorescein diacetate (DCF2DA) using confocal microscopy. The left side shows the ROS generation image of the gentamicin group which was suppressed by capsazepine and ruthenium red. The right side shows the Nomarski images of the corresponding cells on the left.

### DEFINITIONS

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenine. However, when a U is denoted in the context of the present invention, the ability to substitute a T is implied, unless otherwise stated. Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity.

The term “expression cassette” is used to define a nucleotide sequence containing regulatory elements operably linked to a coding sequence that result in the transcription and translation of the coding sequence in a cell.

The term “expression vector” refers to both viral and non-viral vectors comprising a nucleic acid expression cassette.

The term “gene” refers to a nucleic acid (e.g., DNA) sequence that comprises coding sequences necessary for the production of a polypeptide or precursor or RNA (e.g., tRNA, siRNA, rRNA, etc.). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

The terms “homology” or “homologous” when used in the context of nucleic acid or polypeptide sequences refer to sequence identity or similarity between two or more sequences. The degree of sequence identity is generally quantified using percentages, which is calculated based on the number of differing nucleotides or amino acids over the total length of the sequence. As a practical matter, whether any particular nucleic acid molecule is at least, e.g., 70%, 80%, 90%, 95%, 96%, 97% 98%, 99% or 100% identical to the ribonucleotide sequence of a target agent or virus can be determined conventionally using known computer programs such as the Bestfit program (Wisconsin Sequence Analysis Package, Version 8 for Unix, Genetics Computer Group, Madison, Wis.). Bestfit uses the local homology algorithm of Smith and Waterman (Advances in Applied Mathematics 2:482-489 (1981)) to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference ribonucleotide sequence and that gaps in homology of up to 5% of the total number of ribonucleotides in the reference sequence are allowed.

A “mammalian promoter” refers to a transcriptional promoter that functions in a mammalian cell that is derived from a mammalian cell, or both.

“NADPH” is an abbreviation for nicotinamide adenine dinucleotide phosphate.

“NOX3” is an abbreviation for NADPH oxidase 3.

By “ototoxic agent” in the context of the present invention is meant a substance that through its chemical action injures, impairs, or inhibits the activity of a cell or tissue component related to hearing, which in turn impairs hearing and/or balance. In the context of the present invention, ototoxicity includes a deleterious effect on the inner ear sensory hair cells. Ototoxic agents that cause hearing impairments include, but are not limited to therapeutic drugs including antineoplastic agents such as vincristine, vinblastine, cisplatin, taxol, or dideoxy-compounds, e.g., dideoxyinosine; salicylates; quinines; diuretics including furosemide and ethocrynic acid; aminoglycosides; polypeptide antibiotics; contaminants in foods or medicinals; environmental or industrial pollutants; solvents including toluene, xylene, metalloproteins including arsenic and cadmium, and large doses of vitamins such as vitamins A, D, or B6. By “exposure to an ototoxic agent” is meant that the ototoxic agent is made available to, or comes into contact with a patient, such as a human. Exposure to an ototoxic agent can occur by direct administration, e.g., by ingestion or administration of a food, medicinal, or therapeutic agent, e.g., a chemotherapeutic agent, by accidental contamination, or by environmental exposure, e.g., aerial or aqueous exposure.

The term “patient”, as used herein, refers to an animal, preferably a mammal. More preferably the patient can be a primate, including non-humans and humans. The terms subject and patient are used interchangeably herein.

“Pharmaceutically acceptable carrier” includes, but is not limited to, a non-toxic solid, semisolid or liquid filler, diluent, encapsulating material or formulation auxiliary of any type, such as liposomes.

The term “plasmid” as used herein, refers to an independently replicating piece of DNA. It is typically circular and double-stranded.

“Small interfering RNA” (siRNA) refers to double-stranded RNA molecules from about 10 to about 30 nucleotides long that are named for their ability to specifically interfere with protein expression. The length of the siRNA molecule is based on the length of the antisense strand of the siRNA molecule.

“Transfection” is the term used to describe the introduction of foreign material such as foreign DNA into eukaryotic cells. It is used interchangeably with “transformation” and “transduction” although the latter term, in its narrower scope refers to the process of introducing DNA into cells by viruses, which act as carriers. Thus, the cells that undergo transfection are referred to as “transfected,” “transformed” or “transduced” cells.

A “therapeutically effective” amount of the inventive compositions can be determined by prevention or amelioration of adverse conditions or symptoms of diseases, injuries or disorders being treated.

“TRPV1” is an abbreviation for transient receptor potential vanilloid type 1. It is also known as vanilloid receptor type 1.

As used herein, a “3′ overhang” refers to at least one unpaired nucleotide extending from the 3′-end of a duplexed RNA strand.

The term “vector” refers to a DNA molecule into which foreign fragments of DNA may be inserted. Generally, they contain regulatory and coding sequences of interest. The term vector includes but is not limited to plasmids, cosmids, phagemids, viral vectors and shuttle vectors.

### DETAILED DESCRIPTION OF THE INVENTION

The present invention relates to methods for reducing or preventing ototoxicity caused by an ototoxic agent, noise, head and/or neck radiation, or tinnitus. In one embodiment, the method of the present invention involves silencing the expression of TRPV1 gene in the ear of a patient, preferably by administration of siRNA directed against TRPV1 mRNA. In another embodiment, the method involves administration of siRNA directed against NOX3 mRNA. In still another embodiment, the method of the present invention involves administration of at least two siRNAs directed against TRPV1 mRNA and NOX3 mRNA. Furthermore, the present invention also relates to a method for preventing or reducing generation of reactive oxygen species in an inner ear of a patient by administering at least one siRNA selected from siRNA directed against TRPV1 mRNA and NOX3 mRNA.

In one embodiment, the ototoxic agent is selected from aminoglycosides and platinum-containing chemotherapeutic agents. Aminoglycosides include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. In preferred embodiments, the aminoglycosides are selected from neomycin, kanamycin, gentamicin, tobramycin and streptomycin. In still other preferred embodiments, the aminoglycosides are selected from neomycin B, kanamycin A, kanamycin B, gentamicin C1, gentamicin C1a, and gentamicin C2.

Platinum-containing chemotherapeutic agents include but are not limited to cisplatin and carboplatin. In one preferred embodiment, the platinum-containing chemotherapeutic agent is cisplatin.

Head and/or neck radiation is generally administered for purposes of treating head and/or neck cancers including but not limited to tumors of the oral cavity, larynx, pharynx, and major salivary glands. In a preferred embodiment, head and/or neck radiation is administered for purposes of treating squamous cell carcinoma of the oral cavity, larynx or pharynx.

### Hearing Loss Evaluation

Tests are known and available for diagnosing hearing impairments. Neuro-otological, neuro-ophthalmological, neurological examinations, and electro-oculography can be used. (Wennmo et al. Acta Otolaryngol (1982) 94:507-15). Sensitive and specific measures are available to identify patients with auditory impairments. For example, tuning fork tests can be used to differentiate a conductive from a sensorineural hearing loss and determine whether the loss is unilateral. An audiometer is used to quantify hearing loss, measured in decibels. With this device the hearing for each ear is measured, typically from 125 to 8000 Hz, and plotted. The speech recognition threshold, the intensity at which speech is recognized as a meaningful symbol, can be determined at various speech frequencies. Speech or phoneme discrimination can also be determined and used as an indicator of sensorineural hearing loss since analysis of speech sounds relies upon the inner ear and the 8^{th} nerve.

Tympanometry can be used to diagnose conductive hearing loss and aid in the diagnosis of those patients with sensorineural hearing loss.

Electrocochleography (i.e., measuring the cochlear microphonic response and action potential of the 8^{th} nerve to acoustic stimuli), and evoked response audiometry (i.e., measuring evoked response from the brainstem to acoustic stimuli) can be used in patients, particularly infants and children or patients with sensorineural hearing loss of obscure etiology. These tests serve a diagnostic function as well as a clinical function in assessing response to therapy.

Sensory and neural hearing losses can be distinguished based on tests for recruitment (an abnormal increase in the perception of loudness or the ability to hear loud sounds normally despite a hearing loss), sensitivity to small increments in intensity, and pathologic adaptation, including neural hearing loss. In sensory hearing loss, the sensation of loudness in the affected ear increases more with each increment in intensity than it does in the normal ear. Sensitivity to small increments in intensity can be demonstrated by presenting a continuous tone of 20 dB above the hearing threshold and increasing the intensity by 1 dB briefly and intermittently. The percentage of small increments detected yields the “short increment sensitivity index” value. High values, 80 to 100%, are characteristic of sensory hearing loss, whereas a neural lesion patient and those with normal hearing cannot detect such small changes in intensity. Pathologic adaptation is demonstrated when a patient cannot continue to perceive a constant tone above threshold of hearing, also known as tone decay. A Bekesy automatic audiometer or equivalent can be used to determine these clinical and diagnostic signs; audiogram patterns of the Type II pattern, Type III pattern and Type IV pattern are indicative of preferred hearing losses suitable for the treatment methods of the invention. As hearing loss can often be accompanied by vestibular impairment, vestibular function can be tested, particularly when presented with a sensorineural hearing loss of unknown etiology.

When possible, diagnostics for hearing loss, such as audiometric tests, should be performed prior to exposure in order to obtain a patient's normal hearing baseline. Upon exposure, particularly to an ototoxic drug or head and/or neck radiation, audiometric tests can be performed, e.g., twice a week and testing should be continued for a period after cessation of the ototoxic drug treatment or head and neck radiation, since hearing loss may not occur until several days after cessation. For example, U.S. Pat. Nos. 5,546,956 and 4,637,402 provide methods for testing hearing and measuring hearing defects that can be used to diagnose the patient and monitor treatment.

siRNA

In some of the embodiments of the present invention, TRPV1 gene expression in the ear is silenced in order to reduce or prevent ototoxicity caused by an ototoxic agent, noise, head and/or neck radiation or tinnitus. TRPV1 gene expression can be silenced by using siRNA directed against TRPV1, antisense nucleotides directed against TRPV1, or other methods (e.g., by the use of inhibitors of p38 mitogen activator protein kinase—MAPK (Puntambekar et al., 2006, J. Neurochem. 95:1680-1703; Ji et al., 2003, Neuron 36:57-68), which can prevent or significantly reduce TRPV1 mRNA or TRPV1 protein expression. The antisense technology is well known in the art. Briefly, a nucleotide sequence generally containing between 19 and 29 nucleotides is used, which is complementary to the sense mRNA sequence of TRPV1. The degree of complementarity generally ranges from about 70% to about 100%. Preferably, complementarity is greater than 80%, more preferably greater than 90%, and even more preferably greater than 95%. The region of TRPV1 mRNA that should be targeted can be readily determined by comparing the efficacy of several antisense sequences designed to complement different regions of TRPV1 mRNA to prevent production of TRPV1 protein. Such experiments can be readily performed without undue experimentation by any of known techniques in the art, such as Western blotting.

In some embodiments, the silencing of the TRPV1 gene is performed by administering a siRNA directed against TRPV1, wherein the siRNA is preferably applied locally to the ear. By way of example, the siRNA can be administered to the round window or intra-tympanically. In another preferred embodiment, a sense strand of the siRNA has a sequence of SEQ ID NO: 1. In still another preferred embodiment, a sense strand of the siRNA against TRPV1 has a sequence of SEQ ID NO: 2. It should be noted that the sequence of SEQ ID NO: 1 differs from SEQ ID NO: 2 by having the first 2 nucleotides at 5′ removed and by containing 2 extra nucleotides at 3′. SEQ ID NO: 2 was described in a publication by Christoph et al., Biochemical and Biophysical Research Communications 350 (2006) 238-243.

In other embodiments, the present invention relates to a method for preventing and/or reducing ototoxicity in a patient caused by an ototoxic agent, noise, head and/or neck radiation or tinnitus, wherein the method comprises administering more than one siRNA directed against TRPV1 mRNA. By way of example, two siRNAs directed against TRPV1 can be administered, such as a combination of siRNAs whose sense sequences include SEQ ID NO: 1 and SEQ ID NO: 2.

In some other embodiments, the present invention relates to a method for preventing and/or reducing ototoxicity in a patient caused by an ototoxic agent, noise or head and neck radiation, wherein the method comprises administering a siRNA directed against NOX3 mRNA to the patient. In one preferred embodiment, a sense strand of the siRNA directed against NOX3 mRNA contains the sequence of SEQ ID NO: 3. There are also commercially available siRNAs directed against NOX3. By way of example, Ambion offers at least four siRNAs directed against NOX3, which target exons 2, 5, 6 or 13. See siRNA ID numbers 23254, 118793, 118794 and 23441, respectively. (http://www.ambion.com/catalog/sirna_search.php?action=pre&sirna_id=118794) Preferably, the siRNA against NOX3 is applied locally to the ear of a patient, e.g., by round window administration.

In additional embodiments, the present invention provides a method for preventing and/or reducing ototoxicity in a patient caused by an ototoxic agent, noise, head and/or neck radiation, or tinnitus, wherein the method comprises administering at least one siRNA selected from siRNAs directed against TRPV1 mRNA and NOX3 mRNA. In one preferred embodiment, the method comprises administering a combination of a siRNA directed against TRPV1 and a siRNA directed against NOX3. In another preferred embodiment, the method comprises administering a combination of siRNAs whose sense strands include SEQ ID NO: 1 and SEQ ID NO: 3. In still another preferred embodiment, the combination of at least two siRNAs comprises siRNAs with sense strands SEQ ID NO: 2 and SEQ ID NO: 3. In another preferred embodiment, a combination of siRNAs having sense strands comprising SEQ ID NO: 1, SEQ ID NO: 2 and SEQ ID NO: 3 is administered. Preferably, the siRNAs against TRPV1 and NOX3 are applied locally to the ear of a patient, e.g., by round window administration or intra-tympanically.

In addition to the above-mentioned ototoxic agents, salicylates (such as asprin and aspirin-containing drugs) and non-steroidal anti-inflammatory drugs (NSAIDs) (such as naproxen, ibuprofen, diclofenac, piroxicam, indomethacin and the like) can also induce temporary hearing loss. Accordingly, the siRNAs of the present invention can also be used to prevent and/or reduce ototoxicity associated with administration of these agents. Preferably, the administration of at least one siRNA of the present invention is started prior to the start of the salicylate or NSAID regimen, and is continued during such regimen, if needed. Thus, at least one siRNA selected from siRNAs directed against TRPV1 and NOX3 can be used to treat and/or prevent ototoxicity associated with the administration of NSAIDs or salicylates.

As known in the art, the siRNA contains an antisense strand and a sense strand which form an RNA duplex. Thus, the siRNAs of the present invention contain either the TRPV1 antisense and sense sequences or NOX3 antisense and sense sequences.

Thus, in one embodiment of the invention, the antisense RNA sequence is at least 70% complementary to an RNA sequenced from either TRPV1 or NOX3. In other preferred embodiments, the TRPV1 and NOX3 antisense sequences are at least, 80, 90, 95, or 100% complementary to TRPV1 mRNA and NOX3 mRNA sequences, respectively. It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

One skilled in the art can readily determine substitutions and/or mutations in antisense RNA which result in such complementarity. By way of example, if the NOX3 mRNA sequence (sense) and antisense sequences are each 20 nucleotides long, the antisense sequence is 90% complementary to the sense mRNA sequence if 18 out of the 20 nucleotides in the antisense strand can base pair with the nucleotides in the NOX3 mRNA. By way of another example, if the TRPV1 mRNA sequence (sense) and antisense sequences are each 25 nucleotides long, the antisense sequence is 80% complementary to the sense mRNA sequence if 20 out of the 25 nucleotides in the antisense strand can base pair with the nucleotides in the TRPV1 mRNA. When making such substitutions, considerations such as where they are introduced and whether they are dispersed throughout the sequence or occur together can affect the efficacy of the siRNA. By way of example, it is known in the art that substitutions in the center of the molecule tend to affect the efficacy to a greater degree than the substitutions at either end of the molecule. Similarly, two or more contiguous substitutions tend to affect the ability of the antisense RNA to bind to a target molecule to a greater degree than two or more mutations situated throughout the antisense sequence. In one embodiment, the substitutions are introduced such that there are regions of at least 3, more preferably of at least 4, and even more preferably of at least 5 contiguous unmutated nucleotides between each substitution.

The following features are not required but can also be considered when determining which substitutions to make: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand.

In addition, if a substitution results in a potential siRNA target sequence with one or more of the following criteria, such sequence may be less likely to function as siRNA: (1) sequence comprising a stretch of 4 or more of the same base in a row; (2) sequence comprising homopolymers of Gs; (3) sequence comprising triple base motifs (e.g., GGG, CCC, AAA, or TTT); (4) sequence comprising stretches of 7 or more G/Cs in a row; and (5) sequence comprising direct repeats of 4 or more bases within the candidates resulting in internal fold-back structures. However, such sequences should still be evaluated for the ability to function as siRNA molecules.

Accordingly, one of ordinary skill in the art can determine without undue experimentation which substitutions to make in order to achieve the desired complementarity between the sense RNA and antisense RNA.

In addition to substitutions, one or more nucleotides can be chemically modified, e.g., for purposes of reducing immunostimulatory effect of siRNA sequences. In some embodiments, the chemically modified siRNA comprises modified nucleotides including, but not limited to, 2′-O-methyl (2′OMe) nucleotides, 2′-deoxy-2′-fluoro (2′F) nucleotides, 2′-deoxy nucleotides, 2′-O-(2-methoxyethyl) (MOE) nucleotides, locked nucleic acid (LNA) nucleotides, and mixtures thereof. In preferred embodiments, the modified siRNA comprises 2′OMe nucleotides (e.g., 2′OMe purine and/or pyrimidine nucleotides) such as, for example, 2′OMe-guanosine nucleotides, 2′OMe-uridine nucleotides, 2′OMe-adenosine nucleotides, 2′OMe-cytosine nucleotides, and mixtures thereof. The modified siRNA can comprise modified nucleotides in one strand (i.e., sense or antisense) or both strands of the double-stranded region of the siRNA.

In one embodiment, the antisense RNA of the siRNA directed against TRPV1 is from about 19 to about 29 nucleotides long. In another embodiment, the antisense RNA of the siRNA directed against NOX3 is from about 19 to about 29 nucleotides long. Preferably, the antisense RNA of the siRNA molecules of the present invention, namely TRPV1 siRNA or NOX3 siRNA is 20-28 nucleotides long, and still more preferably 21-25 nucleotides long. Therefore, preferred antisense strands of siRNA molecules are 19, 20, 21, 22, 23, 24, 25, 26, 27 28 or 29 nucleotides in length. The sense strand of the siRNA of the present invention is also from about 19 to about 29 nucleotides long, preferably 20-28 nucleotides long, and still more preferably 21-25 nucleotides long. Therefore, preferred sense strands of siRNA molecules of the present invention are 19, 20, 21, 22, 23, 24, 25, 26, 27 28 or 29 nucleotides in length. In another preferred embodiment, the antisense strand is of the same length as the sense strand.

In one embodiment, the sense and antisense strands of the present siRNA are composed of two complementary, single-stranded RNA molecules. In another embodiment, the sense and antisense strands are encoded by a single molecule in which two complementary portions are base-paired and are covalently linked by a single-stranded “hairpin” area. Without wishing to be bound by any theory, it is believed that the hairpin area of the latter type of siRNA molecule is cleaved intracellularly by the “Dicer” protein (or its equivalent) to form an siRNA of two individual base-paired RNA molecules (see Tuschl, T. (2002), supra). Thus, siRNAs against TRPV1 and NOX3 can either be composed of two single-stranded RNA molecules or can be composed of a single molecule in which the sense and antisense strands are separated by a hairpin.

The siRNA of the present invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion. Amino acid deletions, substitutions or additions can be carried out by a site-specific mutagenesis method which is a well known technique. See, for example, Molecular Cloning, A Laboratory Manual, Second Edition, Cold Spring Harbor Laboratory Press (1989); Current Protocols in Molecular Biology, Supplement 1 to 38, John Wiley & Sons (1987-1997); Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci. USA, 82, 488 (1985); Proc. Natl. Acad. Sci., USA, 81, 5662 (1984); Science, 224, 1431 (1984); PCT WO85/00817 (1985); Nature, 316, 601 (1985); and the like.

One or both strands of the siRNA of the invention can also comprise a 3′ overhang. Thus in some embodiments, the siRNA directed against TRPV1 or NOX3 comprises at least one 3′ overhang of from 1 to about 6 nucleotides (which includes ribonucleotides or deoxynucleotides) in length, preferably from 1 to about 5 nucleotides in length, more preferably from 1 to about 4 nucleotides in length, and even more preferably from about 2 to about 4 nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is 2 nucleotides in length. For example, each strand of the siRNA of the invention can comprise 3′ overhangs of dithymidylic acid (“TT”) or diuridylic acid (“uu”).

In order to enhance the stability of the present siRNA, the 3′ overhangs can be also stabilized against degradation. In one embodiment, the overhangs are stabilized by including purine nucleotides, such as adenosine or guanosine nucleotides. Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

siRNA Preparation

siRNA can be prepared in a number of ways, such as by chemical synthesis, T7 polymerase transcription, or by treating long double stranded RNA (dsRNA) prepared by one of the two previous methods with Dicer enzyme. Dicer enzyme creates mixed populations of dsRNA from about 21 to about 23 base pairs in length from dsRNA that is about 500 base pairs to about 1000 base pairs in size. Dicer can also effectively cleave modified strands of dsRNA, such as 2′ fluoro-modified dsRNA. The Dicer method of preparing siRNAs can be performed using a Dicer siRNA Generation Kit available from Gene Therapy Systems (San Diego, Calif.).

In one preferred embodiment, the siRNA directed against TRPV1 or NOX3 is synthetically produced. By way of example and not of limitation, the siRNAs of the present invention are chemically synthesized using appropriately protected ribonucleotides phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo. USA), Pierce Chemical (part of Perbio Science, Rockford, Ill., USA), Glen Research (Sterling, Va. USA), ChemGenes (Ashland, Mass. USA) and Cruachem (Glasgow, UK). The siRNA of the present invention can be a recombinantly produced RNA. A number of siRNAs are commercially available and can be purchased from vendors such as Ambion and Santa Cruz Biotechnology, Inc.

A variety of different vectors can be employed for producing siRNAs by recombinant techniques. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector can be used as long as it is replicable and viable in a desired host.

The siRNA of the present invention can be expressed from a recombinant plasmid either as two separate, complementary RNA molecules, or as a single RNA molecule with two complementary regions.

Selection of plasmids suitable for expressing siRNA of the invention, methods for inserting nucleic acid sequences for expressing the siRNA into the plasmid, and methods of delivering the recombinant plasmid to the cells of interest are within the skill in the art. See, for example, Tuschl, T. (2002), Nat. Biotechnol, 20: 446-448, Brummelkamp T R et al. (2002), Science 296: 550-553 Miyagishi M et al, (2002), Nat. Biotechnol. 20: 497-500; Paddison P J et al. (2002). Genes Dev. 16: 948-9578; Lee N S et al. (2002) Nat. Biotechnol, 20: 500-505; and Paul C P et al. (2002). Nat. Biotechnol. 20: 505-508. Selection of viral vectors suitable for use in the present invention are also within the skill in the art. See, for example, Dornburg R (1995), Gene Therap. 2: 301-310; Eglitis M A (1988), Biotechniques 6: 608-1614; Miller A D (1990), Hum Gene Therap. 1: 5-14; and Anderson W F (1998), Nature 392: 25-30.

The appropriate DNA segment may be inserted into the vector by a variety of procedures. In general, the DNA sequence is inserted into an appropriate restriction endonuclease site(s) by procedures known in the art, which can be performed without undue experimentation by a skilled artisan. The DNA segment in the expression vector is operatively linked to an appropriate expression control sequence(s) (promoter) to direct siRNA synthesis. Suitable eukaryotic promoters include the CMV immediate early promoter, the herpes simplex virus (HSV) thymidine kinase promoter, the early and late SV40 promoters, the promoters of retroviral long terminal repeats (LTRs), such as those of the Rous Sarcoma Virus (RSV), and metallothionein promoters, such as the mouse metallothionein-I promoter. Preferably, the promoters of the present invention are from the type III class of RNA polymerase III promoters. More preferably, the promoters are selected from the group consisting of the U6 and H1 promoters. In still another preferred embodiment, the promoter is a U6 promoter.

The promoters of the present invention may also be inducible, in that expression may be turned “on” or “off.” For example, a tetracycline-regulatable system employing the U6 promoter may be used to control the production of siRNA. Additionally, promoters which are tissue specific or respond to a particular stimulus can also be used. By way of example and not of limitation, tissue specific promoters include promoters which are active in the liver, such as, e.g., albumin promoter. Promoters which respond do a particular stimulus include, e.g., heat shock protein promoters, and Tet-off and Tet-on promoters.

In addition, the expression vectors preferably contain one or more selectable marker genes to, provide a phenotypic trait for selection of transformed host cells such as dihydrofolate reductase or neomycin resistance for eukaryotic cell culture, or tetracycline or ampicillin resistance.

In one embodiment, the invention provides a vector, wherein the DNA segment encoding the sense strand of the RNA polynucleotide is operably linked to a first promoter and where the DNA segment encoding the antisense (opposite) strand of the RNA polynucleotide molecule is operably linked to a second promoter. In other words, each strand of the RNA polynucleotide is independently expressed. Furthermore, the promoter driving expression of each strand can be identical or each one may be different from the other promoter. In another embodiment, the vector used to express a siRNA of the present invention can include opposing promoters. For example, the vector can contain two U6 promoters on either side of the DNA segment encoding the sense strand of the RNA polynucleotide and placed in opposing orientations, with or without a transcription terminator placed between the two opposing promoters. The U6 opposing promoter construct is similar to the T7 opposing promoter construct as described in, e.g., Wang, Z. et al., J. Biol. Chem. 275: 40174-40179 (2000). In another embodiment, the DNA segments encoding both strands of the RNA polynucleotide are under the control of a single promoter. In one embodiment, the DNA segments encoding each strand are arranged on the vector with a “loop” region interspersed between the two DNA segments, where transcription of the DNA segments and loop region creates one RNA transcript. The single transcript will, in turn, anneal to itself creating a “hairpin” RNA structure capable of inducing RNAi. The “loop” of the hairpin structure is preferably from about 4 to about 12 nucleotides in length. More preferably, the loop is 9 nucleotides in length.

Any viral vector capable of accepting the coding sequences for the siRNA molecule(s) to be expressed can be used, for example vectors derived from adenovirus (AV); adeno-associated virus (AAV); retroviruses (e.g. lentiviruses (LV), Rhabdoviruses; herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like. In one embodiment, preferred viral vectors are those derived from lentiviruses.

The vector containing the appropriate DNA sequence as described herein, as well as an appropriate promoter or control sequence, can be employed to transform an appropriate host to permit the host to express the siRNA. Appropriate cloning and expression vectors for use with prokaryotic and eukaryotic hosts are described by Sambrook, et al., Molecular Cloning: A Laboratory Manual, Second Edition, Cold Spring Harbor, N.Y. (1989). In one embodiment, the cells used to produce siRNAs of the present invention are HEK 293T cells.

Detection of siRNA

The ability of an siRNA containing a given target sequence to cause RNA-mediated degradation of the target mRNA can be evaluated using standard techniques for measuring the levels of RNA or protein in cells. Methods for the determination of mRNA expression levels are known in the art and comprise Real Time PCR, Northern blotting and hybridization on microarrays or DNA chips.

Additionally, the methods described in the above sections related to hearing tests can also be used to assess the efficacy of siRNAs to prevent or reduce hearing loss.

siRNA Delivery

In the present methods, the present siRNA can be administered to the subject either as naked siRNA, in conjunction with a delivery reagent, or as a recombinant plasmid or viral vector which expresses the siRNA.

In some preferred embodiments, at least one siRNA of the present invention is administered locally to the ear, e.g., by application to the round window. Delivery of therapeutic agents in a controlled and effective manner with respect to tissue structures of the inner ear, for example, those portions of the ear contained within the temporal bone which is the most dense bone tissue in the human body, is known in the art. Exemplary inner ear tissue structures of primary importance include but are not limited to the cochlea, the endolymphatic sac/duct, the vestibular labyrinth, and all of the compartments which include these components. Access to the foregoing inner ear tissue regions is typically achieved through a variety of structures, including but not limited to the round window membrane, oval window/stapes footplate, the annular ligament, and systemically. In some preferred embodiments, the siRNA directed against TRPV1, the siRNA directed against NOX3 or a combination thereof is administered locally to the ear by administration to the round window membrane.

The siRNA is applied to the round window membrane by one of several methods, such as: 1) direct application of the siRNA, in a solution or otherwise, by surgical exposure of the round window membrane and administering the siRNA with a syringe and blunt needle, or 2) with a wick or catheter to direct the siRNA to the round window membrane by using an endoscope or a surgical microscope to guide the application. By way of example, the siRNA of the present invention is added as a drop of fluid on the round window using a syringe, following which it diffuses slowly into the perilymph over a 1 hour period.

Intratympanic administration can be performed by injection through an intact tympanic membrane or through a ventilation tube surgically inserted through the tympanic membrane; or as an otic drop solution, applied to the ear canal and allowed to enter the middle ear through an existing perforation of the eardrum or through a ventilation tube placed in the tympanic membrane. The entry of the solution into the middle ear which allows access to the round window membrane is assisted by a procedure called “tragal pumping” which simply involves pushing the tragus of the ear gently in a medial direction to push the solution through a ventilation tube into the middle ear. The tragus is the portion of the external, ear in front of the ear canal which consists of skin-covered cartilage. This procedure is routinely used to administer ear drops for middle ear infections in patients with a ventilation tube in place or a perforation of the ear drum. By way of example, repeated applications of the siRNA can be carried out with a surgically implanted catheter placed under local anesthesia.

When it is administered as naked siRNA, the delivery to the cells can be achieved, e.g., by electroporation or gene gun method. Both are well-known in the art, and described, e.g., in Sambrook and Russell, Molecular Cloning: A Laboratory manual, third edition, Cold Spring Harbor Laboratory Press America, 2001.

Suitable delivery reagents for administration in conjunction with the present siRNA include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the siRNA to a particular tissue, and can also increase the blood half-life of the siRNA. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al. (1980), Ann. Rev. Biophys. Bioeng. 9: 467; and U.S. Pat. Nos. 4,235,871, 4,501,728, 4,837,028, and 5,019,369. In one embodiment, the liposomes encapsulating the present siRNA comprises a ligand molecule that can target the liposome to the ear.

The liposomes encapsulating the present siRNA can be modified so as to avoid clearance by the mononuclear macrophage and reticuloendothelial systems, for example by having opsonization-inhibition moieties bound to the surface of the structure. In one embodiment a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016. Liposomes modified with opsonization-inhibition moieties thus remain in the circulation much longer than unmodified liposomes. For this reason, such liposomes are sometimes called “stealth” liposomes.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate synthetic polymers such as polyacrylamide or poly N-vinyl pyrrolidone; linear, branched, or dendrimeric polyamidoamines; polyacrylic acids; polyalcohols, e.g., polyvinylalchohol and polyxylitol to which carboxylic or amino groups are chemically linked, as well as gangliosides, such as ganglioside GM1.

Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic, acid, carageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor and then bound to a membrane. Similarly, a dextran polymer can be derivatized with a stearylamine lipid-soluble anchor via reductive amination using NaCN)BH3 and a solvent mixture such as tetrahydrofuran and water in a 30:12 ratio at 60° C.

Recombinant plasmids which express siRNA of the invention are discussed above. Such recombinant plasmids can also be administered directly or in conjunction with a suitable delivery reagent, including the Mirus Transit LT1 lipophilic reagent; lipofetin; lipofectamine; cellfectin; polycations (e.g., polylysine) or liposomes. Recombinant viral vectors which express siRNA of the invention are also discussed above, and methods for delivering such vectors to the ear of a patient are within the skill in the art.

Suitable enteral administration routes include oral, sublingual, rectal or intranasal delivery.

Suitable parenteral administration routes include intravascular administration (e.g. intravenous bolus injection, intravenous infusion, intraarterial bolus injection, intraarterial infusion and catheter instillation into the vasculature); peri- and intra-tissue administration; subcutaneous injection or deposition including subcutaneous infusion (such as by osmotic pumps); and inhalation. In a preferred embodiment, injections or infusions of the siRNA are given in the ear or near it.

### Pharmaceutical Compositions

The siRNA of the invention can be formulated as pharmaceutical compositions prior to administering to a subject, according to techniques known in the art. Pharmaceutical compositions of the present invention are characterized as being at least sterile and pyrogen-free. Methods for preparing pharmaceutical compositions of the invention are within the skill in the art, for example as described in Remington's Pharmaceutical Science, 17th ed., Mack Publishing Company, Easton, Pa., (1985).

The present pharmaceutical formulations comprise at least one siRNA of the invention (e.g., 0.1 to 90% by weight), or a physiologically acceptable salt thereof, mixed with a pharmaceutically acceptable carrier. Preferred physiologically acceptable carriers are water, buffered water, saline solutions (e.g., normal saline or balanced saline solutions such as Hank's or Earle's balanced salt solutions), 0.4% saline, 0.3% glycine, hyaluronic acid and the like. In some embodiments, the pharmaceutical composition comprises a siRNA directed against TRPV1. In one embodiment, a sense strand of the siRNA comprises SEQ ID NO: 1. In another embodiment, a sense strand of the siRNA directed against TRPV1 comprises SEQ ID NO: 2. In another embodiment, the pharmaceutical composition of the present invention comprises at least two siRNA directed against TRPV1, such as, e.g., at least two siRNAs with sense strands comprising SEQ ID NO: 1 and SEQ ID NO: 2. The pharmaceutical composition of the present invention can also include at least one siRNA directed against NOX3. In one embodiment, such pharmaceutical composition can include, e.g., a siRNA with a sense strand comprising SEQ ID NO: 3. In other embodiments, the pharmaceutical composition of the present invention can include a combination of siRNAs directed against TRPV1 mRNA and NOX3 mRNA, wherein the sense strands are, e.g., SEQ ID NO: 1 and SEQ ID NO: 3, and SEQ ID NO: 2 and SEQ ID NO: 3, or SEQ ID NO: 1, SEQ ID NO: 2 and SEQ ID NO: 3.

The pharmaceutical composition of the present invention can be administered orally, nasally, parenterally, intrasystemically, intraperitoneally, topically (as by drops or transdermal patch), bucally, sublingually or as an oral or nasal spray. In one preferred embodiment, the pharmaceutical composition of the present invention is administered locally, such as topically. In still another preferred embodiment, the pharmaceutical composition is given by round window administration.

A pharmaceutical composition of the present invention for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

In some cases, to prolong the effect of the drugs, it is desirable to slow the absorption from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle. Prolonged absorption of the injectable pharmaceutical form can be brought about by the inclusion of agents which delay absorption such as aluminum monostearate and gelatin.

Solid dosage forms for oral administration include, but are not limited to, capsules; tablets, pills, powders, and granules. In such solid dosage forms, the active compounds are mixed with at least one item pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The pharmaceutical compositions of the present invention can also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide., oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the siRNAs of the present invention, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof. Alternatively, the composition can be pressurized and contain a compressed gas, such as nitrogen or a liquefied gas propellant. The liquefied propellant medium and indeed the total composition are preferably such that the active ingredients do not dissolve therein to any substantial extent. The pressurized composition can also contain a surface active agent. The surface active agent can be a liquid or solid non-ionic surface active agent or can be a solid anionic surface active agent. It is preferred to use the solid anionic surface active agent in the form of a sodium salt.

Pharmaceutical compositions of the invention can also comprise conventional pharmaceutical excipients and/or additives. Suitable pharmaceutical excipients include stabilizers, antioxicants, osmolality adjucting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example calcium chloride, calcium ascorbate, calcium gluconate or calcium lactate).

Pharmaceutical compositions comprising the siRNAs of the present can include penetration enhancers in order to enhance the alimentary delivery of the oligonucleotides. Penetration enhancers may be classified as belonging to one of five broad categories, i.e., fatty acids, bile salts, chelating agents, surfactants and non-surfactants (Lee et al., Critical Reviews in Therapeutic Drug Carrier Systems 1991, 8, 91-192; Muranishi, Critical Reviews in Therapeutic Drug Carrier Systems 1990, 7, 1-33). One or more penetration enhancers from one or more of these broad categories may be included. Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (1-monooleoyl-rac-glycerol), dilaurin, caprylic acid, arachidonic acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee et al., Critical Reviews in Therapeutic Drug Carrier Systems 1991, page 92; Muranishi, Critical Reviews in Therapeutic Drug Carrier Systems 1990, 7, 1; El-Hariri et al., J. Pharm. Pharmacol. 1992 44, 651-654).

Chelating agents include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) [Lee et al., Critical Reviews in Therapeutic Drug Carrier Systems 1991, page 92; Muranishi, Critical Reviews in Therapeutic Drug Carrier Systems 1990, 7, 1-33; Buur et al., J. Control Rel. 1990, 14, 43-51).

Surfactants include, for example, sodium lauryl sulfate, polyoxyethylene-9-lauryl ether and polyoxyethylene-20-cetyl ether (Lee et al., Critical Reviews in Therapeutic Drug Carrier Systems 1991, page 92); and perfluorochemical emulsions, such as FC43 (Takahashi et al., J. Pharm. Phamacol. 1988, 40, 252-257).

Non-surfactants include, for example, unsaturated cyclic ureas, 1-alkyl- and 1-alkenylazacyclo-alkanone derivatives (Lee et al., Critical Reviews in Therapeutic Drug Carrier Systems 1991, page 92); and non-steroidal anti-inflammatory agents such as diclofenac sodium, indomethacin and phenylbutazone (Yamashita et al., J. Pharm. Pharmacol. 1987, 39, 621-626).

Prevention of the action of microorganisms can be ensured by the inclusion of various antibacterial and antifungal agents, for example, paraben, chlorobutanol, phenol, sorb acid, and the like.

One of ordinary skill in the art will appreciate that effective amounts of the agents of the invention can be determined empirically and can be employed in pure form or, where such forms exist, in pharmaceutically acceptable salt, ester or prodrug form. The agents can be administered to a patient in order to prevent and/or reduce ototoxicity resulting from ototoxic agents, noise or head and neck radiation as pharmaceutical compositions in combination with one or more pharmaceutically acceptable excipients. It will be understood that, when administered to a human patient, the total daily usage of the agents or composition of the present invention will be decided by the attending physician within the scope of sound medical judgment. The specific therapeutically effective dose level for any particular patient will depend upon a variety of factors: the type and degree of the cellular or physiological response to be achieved; activity of the specific agent or composition employed; the specific agents or composition employed; the age, body weight, general health, sex and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

One skilled in the art can also readily determine an appropriate dosage regimen for administering the siRNA of the invention to a given subject. For example, the siRNA can be administered to the subject once, such as by a single injection or deposition. Alternatively, the siRNA can be administered to a subject multiple times daily or weekly. For example, the siRNA can be administered to a subject once weekly for a period of from about three to about twenty-eight weeks, more preferably from about seven to about ten weeks. The administration regimen will also depend on the cause of ototoxicity and exposure of the patient to any of the ototoxic agents, noise or head and neck radiation. The siRNAs of the present invention can either be administered prophylactically, e.g., before starting the aminoglycoside regimen, during exposure of the patient to the ototoxic environment, following such exposure, or a combination thereof. By way of example, a siRNA directed against TRPV1 or NOX3 can be administered prior to the start of cisplatin treatment, during such treatment, and optionally following such treatment. By way of another example, a siRNA against TRPV1 or NOX3 can be administered prior to the start of an aminoglycoside treatment, during such treatment, and optionally following such treatment. When administered after the drug treatment or noise exposure, it is preferable that the siRNA of the present invention be administered within a reasonable time thereafter, e.g., such as a few weeks.

In another embodiment, the siRNA of the present invention can be administered in combination with one or more agents used to treat ototoxicity, such as sodium thiosulfate, D- or L-methionine, diethyldithiocarbamate, methylthiobenzoic acid, lipoic acid, N-acetylcysteine, thiopronine, glutathione ester, and amifostine. Thus, by way of example, a siRNA directed against TRPV1 or NOX3 can be administered in combination with D-methionine.

In another embodiment, the siRNA of the present invention can be used to prevent or reduce generation of reactive oxygen species in the inner ear of a patient. As described above, at least one siRNA selected from siRNAs directed against TRPV1 and NOX3 can be used. Furthermore, since the generation of reactive oxygen species plays a role in tinnitus, the present invention provides a method for treating a patient suffering from tinnitus and a method for preventing and/or reducing ototoxicity in a patient suffering from or at risk for developing ototoxicity associated with tinnitus. For methods of treating tinnitus, the selection of siRNAs, their administration and dosage is the same or can be determined as described above.

### Treatment of Nephrotoxicity

Nephrotoxicity can be induced by aminoglycoside antibiotics and by platinum-containing drugs such as cisplatin. As can be expected, nephrotoxicity has important consequences for the patient, with potential permanent loss of 50% or more of normal renal function (Kemp, et al. J. Clin. Oncology, 14:2101-2112, 1996). This can produce serious disability, requiring the need for dialysis in severe cases, and early mortality. It also has important consequences for the ability of the patient to be safely treated with medications such as antibiotics that are themselves renally toxic or require adequate renal function for elimination from the body. Thus, it is contemplated that siRNAs against TRPV1 and NOX3 can also be used to treat nephrotoxicity in a patient, wherein the nephrotoxicity is caused by administration of aminoglycosides or platinum-containing chemotherapeutic agents. In this embodiment, at least one siRNA of the present invention can be administered by any method known in the art that will efficiently result in the presence of the siRNA(s) in the kidney. By way of example, at least one siRNA of the present invention can be administered intravenously or intraperitoneally. In one preferred embodiment, at least one siRNA directed against TRPV1 is administered, e.g., intravenously. In another preferred embodiment, at least one siRNA directed against NOX3 is administered, e.g., intravenously. In still another preferred embodiment, a combination of at least one siRNA directed against TRPV1 and at least one siRNA directed against NOX3 is administered, e.g., intravenously.

### General Methods

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-interscience; Ausubel, F. M. (1989), Short Protocols in Molecular Biology: A Compendium of Methods from Current Protocols in Molecular Biology, Greene Pub. Associates and Wiley-interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Idenshi Donyu & Hatsugenkaiseki Jikkenho [Experimental Method for Gene introduction & Expression Analysis]”, Yodo-sha, 1997; and the like. Relevant portions (or possibly the entirety) of each of these publications are herein incorporated by reference.

Any technique may be used herein for introduction of a nucleic acid molecule into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] Experimental Method for Gene introduction & Expression Analysis”, Yodo-sha, 1997; and the like. Gene introduction can be confirmed by method as described herein, such as Northern blotting analysis and Western blotting analysis, or other well-known, common techniques.

Having described the invention in detail, it will be apparent that modifications and variations are possible without departing the scope of the invention defined in the appended claims. Furthermore, it should be appreciated that all examples in the present disclosure, while illustrating the invention, are provided as non-limiting examples and are, therefore, not to be taken as limiting the various aspects of the invention so illustrated.

### Examples

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

Methods

Animal procedures and sample collection: Male Wistar rats were used for this study. Pre-ABR's were performed 2-3 days before round window application of siTRPV1 or PBS. 48 hrs post surgery, cisplatin (13 mg/kg) was administered by intraperitoneal injections over a period of 30 min and sacrificed at 24, 48 or 72 h following treatment. The cochleas were dissected and used for the preparation of total RNA or total protein extracts, or perfused with 2.5% glutaraldehyde for morphological studies by Scanning Electron Microscopy (SEM's) or 4% paraformaldehyde for immunocytochemistry.

Measuring Evoked Potentials

Auditory brainstem responses were measured pre-surgery and 72 his post cisplatin treatment as described in (Tanaka, et al. 2003, pH paper). Animals were tested with a stimulus intensity series that was initiated at 10 dB SPL and reached a maximum at 90 dB SPL. The stimulus intensity levels were increased in 10 dB increments, and the evoked ABR waveforms were observed on a video monitor. The auditory stimuli included tone bursts at 2, 4, 8, 16 and 32 kHz with a 10 msec plateau and a 1 msec rise/fall time presented at a rate of 5 per second. Threshold was defined as the lowest intensity capable of evoking a reproducible, visually detectable response with two distinct waveforms and a minimum amplitude of 0.5 μV. The pretreatment ABR thresholds were compared to post-treatment thresholds and the differences were evaluated for statistical significance using the Student's t-test.

Morphological Studies: Scanning Electron Microscopy

Immediately after completion of follow-up ABRs, deeply sedated rats were euthanized, their cochleae harvested and processed as described in (Kamimura et al, 1999). Sputter coated cochleae were then viewed and photographed with a Hitachi S-500 scanning electron microscope (Hitachi Ltd., Tokyo, Japan).

Processing of Cochlea for Immunocytochemistry

Cochleae perfused with 4% paraformaldehyde were processed for decalcification and sectioning as described in (Dunaway et al., 2003). TRPV1-1 antibody was diluted 1:100 and samples were incubated for 1 h at 37° C. incubator. Secondary antibody used was goat anti-rabbit IgG conjugated to horse radish peroxidase (Santa Cruz Biotechnology, Inc, Santa Cruz, Calif.) which was diluted 1:200. ABC staining system (Santa Cruz Biotechnology), which included a diaminobenzadine as a peroxidase substrate, was used for visualization of protein expression. Slides were imaged using Scion Imaging system (Frederick, Md.).

Hair Cell Count

Hair cell counts were performed using a modified version of the method used by (Korver et al. 2002). Two representative areas of the basal turn and hook portion were photographed. In each area, inner or outer hair cells were counted in an area that was 10 pillar cell heads in length.

The results were shown, as the average survival percentage rates compared to the control group. Statistical analysis was performed using Student's t-test.

Cell Cultures

Immortalized organ of Corti cells derived from the mouse, UB/OC-1 cells, were obtained from Dr. Matthew Holley (Institute of Molecular Physiology, Addison Building, Western Bank, Sheffield, UK) and cultured in RPMI 1640 supplemented with 10% Fetalclone II (Hyclone, Logan, Utah) serum and penicillin-streptomycin. Cultures were grown in a 33° C. incubator in 10% CO2.

Reagents

The various reagents: cisplatin, diphenyleneiodonium (DPI), AEBSF Capsezapine, Capsaicin, Ruthinium red, TR1 reagent and, 2-bis(2-aminophenoxy)ethane-N,N,N′,N′-tetraacetic acid (BAPTA) were purchased from (Sigma-aldrich, St. Louis, Mo.). Frag-EL DNA fragmentation kit and H2DCFDA dye was purchased from (Calbiochem, San Diego, Calif.). TRPV1 antibody (Neuromics, Edina, Minn.) and secondary goat anti rabbit antibody was purchased from Santa Cruz Biotechnology (Santa Cruz, Calif.).

RNA Isolation

RNA was isolated by adding 1 ml TR1 reagent to 100 mg of cochlear or cortical tissue or 0.5 ml TR1 reagent (Sigma, St. Louis, Mo.) per well of each six well plate, according to the manufacturer's instructions.

Real time reverse transcriptase polymerase chain reaction (RT-PCR): One microgram of total RNA was converted to cDNA using iScript cDNA Synthesis Kit (Bio-Rad, Hercules, Calif.), and qRTPCR was performed as described by (Mukherjea et al, 2006). Gene specific primer pairs were used for the various reactions and mRNA expression levels were normalized to the levels of GAPDH house keeping gene.

Oligonucleotides

The rodent set of primers and siRNA were based on the homologous sequences in the rat and mouse cDNA sequences. The primers were purchased from Sigma Genosys (St. Louis, Mo.). Purified siRNA duplexes were purchased from Qiagen (Valencia, Calif.). Primers were as follows: Rodent NOX3 (sense): 5′-GTG AAC AAG GGA AGG CTC AT-3′ (SEQ ID NO: 9) (antisense): 5′-GAC CCA CAG AAG AAC ACG C-3′ (SEQ ID NO: 10), Rodent-GAPDH (sense): 5′-ATG GTG AAG GTC GGT GTG AAC-3′ (SEQ ID NO: 11) (antisense): 5′-TGT AGT TGA GGT CAA TGA AGG-3′ (SEQ ID NO: 12), Rodent TRPV1 (sense): 5′-CAA GGC TGT CTT CAT CAT CC-3′ (SEQ ID NO: 13), (antisense): 5-AGT CCA GTT TAC CTC GTC CA-3′ (SEQ ID NO: 14), Rodent Rac-1 (sense): 5′-ATC AGT TAC ACG ACC AAT GC-3′ (SEQ ID NO: 15), (antisense): 5′-GGG AAA AGC AAA TTA AGA AC-3′ (SEQ ID NO: 16), Rodent gp-91 (sense) 5′-TAA AGG AGT GCC CAG TAC CAA-3′ (SEQ ID NO: 17), (antisense): 5′-AAT CCC TTC TTC TTC ATC TGA-3′ (SEQ ID NO: 18), and Rat p22 (sense): 5′-ACA GGG GGC ATC GTG GCT ACT-3′ (SEQ ID NO: 19), (antisense) 5′-GGA CGT AGT AAT TTC TGG TGA-3′ (SEQ ID NO: 20)

Rodent siNOX3: Target sequence: 5′-AAGGTGGTGAGTCACCCATCT-3′ (SEQ ID NO: 8)

Rodent siTRPV1: Target sequence: 5′-GCGCATCTTCTACTTCAACTT-3′ (SEQ ID NO: 7)

A sense strand of siRNA from Christoph et al. (2006) 5′GCGCAUCUUCUACUUCAACTT-3′ (SEQ ID NO: 2)

A sense strand of siRNA against TRPV1: GGU GGU GAG UCA CCC AUC UdTdT (SEQ ID NO: 1)

A sense strand of siRNA against NOX3: GGU GGU GAG UCA CCC AUC U)dTdT (SEQ ID NO: 3)

Western Blot Analysis

Cochleae were homogenized in ice-cold TRIS 50:10:100. The whole tissue lysate was then used for Western blotting. After transfer to nitrocellulose membrane TRPV1 protein was visualized by chemiluminesence detection (ECL, Amersham, Piscataway, N.J.).

Results

Cisplatin

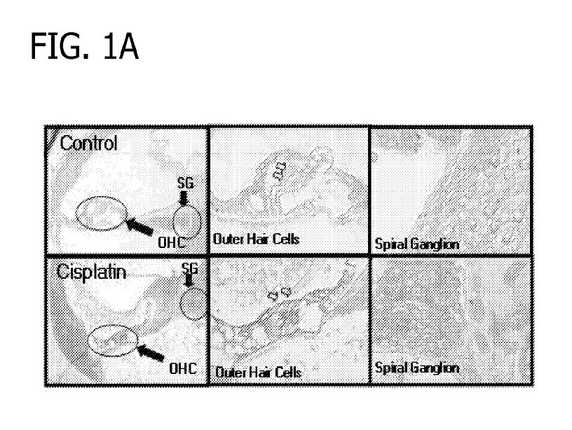

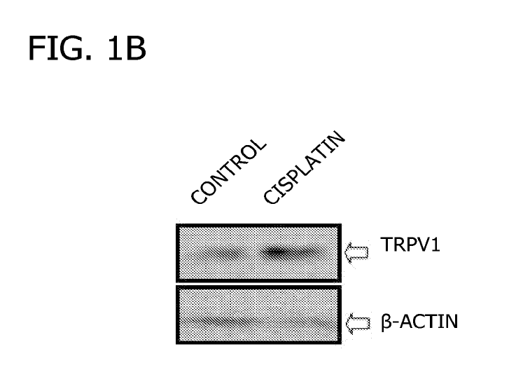

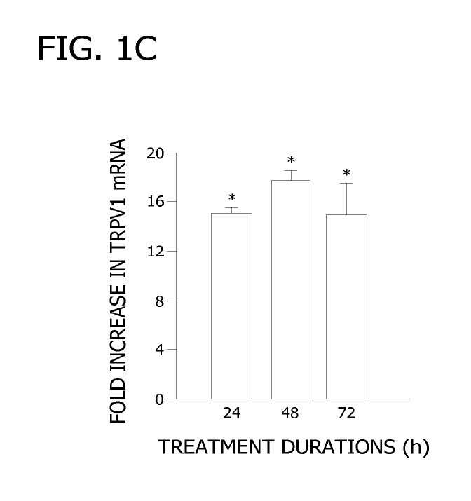

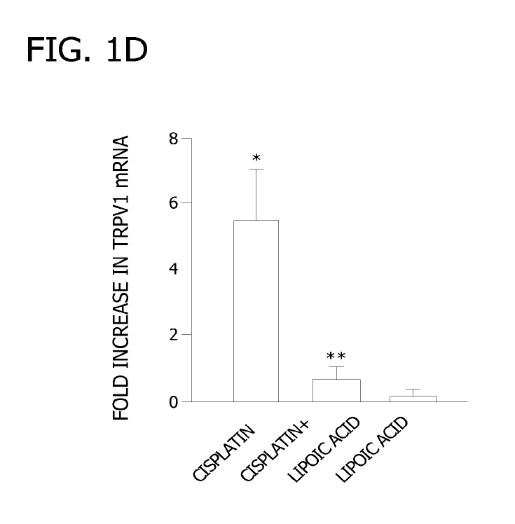

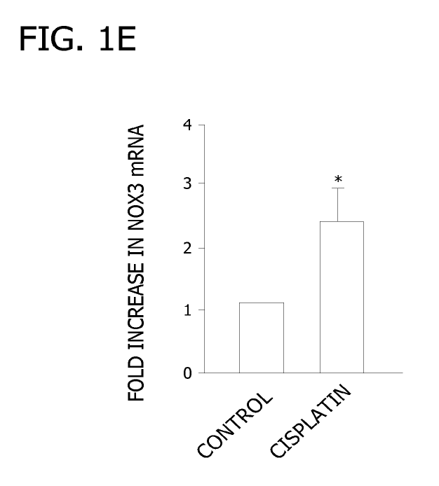

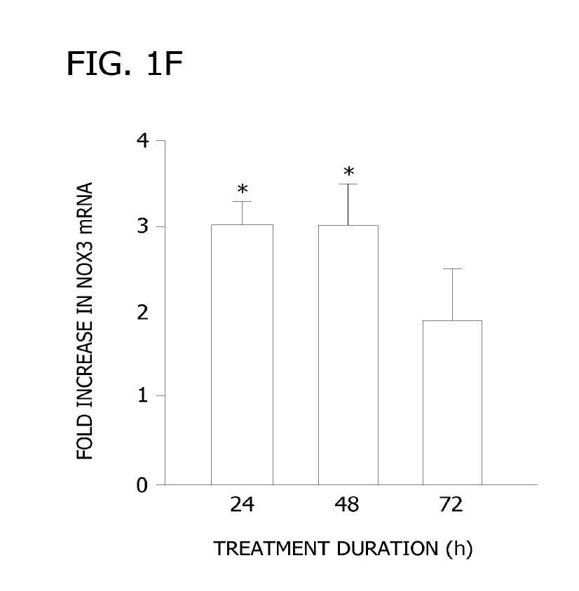

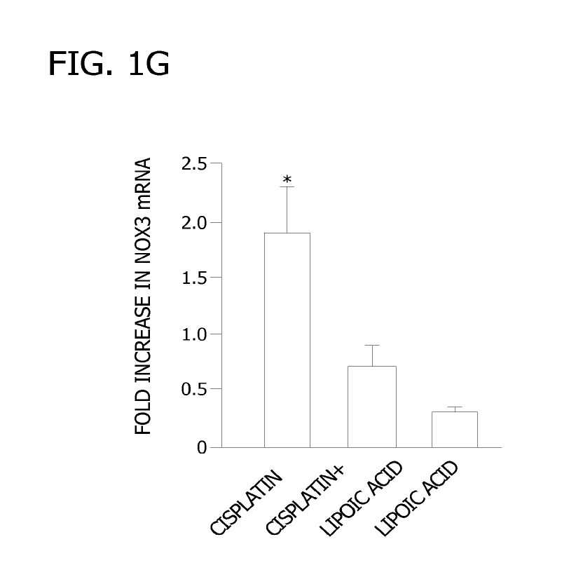

For these studies, male Wistar rats (200-250 g) were administered vehicle or cisplatin (13 mg/kg) by intraperitoneal infusion over a 30 min period. Rats were tested for hearing loss 72 h later using auditory brainstem evoked responses (ABRs). Cisplatin produced a significant elevation in auditory evoked brain stem responses by 20-40 dB over a wide frequency range (8-32 kHz) (as described in Whitworth et al., Biochem Pharmacol., 67, 1801-1807, 2004; Mukherjea et al., Neuroscience, 139, 733-740, 2006). Cochleas obtained from these animals were decalcified, sectioned and processed for TRPV1 immunoreactivity using a polyclonal antibody (as described in Puntambekar et al., 2005). Visualization by confocal microscopy indicated TRPV1 immunoreactivity in the organ of Corti, supporting cells and spiral ganglion cells. Both inner and outer hair cells showed immunolabeling. TRPV1 immunoreactivity, as determined by confocal microscopy, was elevated in hair cells and spiral ganglion cells by 2-3 fold by 72 h following cisplatin treatment (FIG. 1A). No labeling was obtained if the antibody was not added to the immunolabeling mix, suggesting antibody-specific labeling. The increase in immunolabeling observed at 72 h was substantiated by Western blotting studies showing increased TRPV1 protein (˜95 kDa band) in whole cochlear lysates (FIG. 1B). The increases in TRPV1 immunoreactivity were associated with a 15±1-fold increase in TRPV1 transcripts by 24 h following cisplatin treatment, with no further elevations by 48 and 72 h. The increases were 18±1 and 15±3 at 48 h and 72 h, respectively (FIG. 1C). These increases in TRPV1 transcripts preceded morphological changes in hair cell, which are generally observed by 72 h following cisplatin administration (as shown by Ford et al., Hear Res., 111, 143-152, 1997 and Whitworth et al., 2004, supra) and which involve ROS generation (Kopke, Am. J. Otol., 18, 559-571, 1997). To determine whether ROS generation could be implicated in TRPV1 induction, as demonstrated previously (Puntambekar et al., J. Neurochem., 1689-1703, 2005), the effect of the antioxidant, lipoic acid, was tested on cisplatin-induced TRPV1 expression in vivo. Even though the level of induction of TRPV1 by cisplatin (5.5±1.5-fold) was smaller than obtained previously, it was completely abolished in rats pretreated with lipoic acid (FIG. 1D). Lipoic acid also showed a tendency to reduce basal expression of TRPV1 (albeit not statistically significant), implicating ROS in this response.

The NOX3 isoform of NADPH oxidase represents the most abundant form of this enzyme present in the cochlea, whose expression is induced by cisplatin in organotypic cultures (Banfi et al., J. Biol. Chem., 279, 46065-46072, 2004). Results shown in FIG. 1E indicate a small but statistically significant increase in NOX3 transcript in the cochlea by cisplatin. This induction was close to maximum by 24 h, with no further significant change observed by 48 h and a reduction by 72 h (FIG. 1F). The increase in NOX3 by cisplatin was attenuated by lipoic acid, implicating ROS in its induction (FIG. 1G). In addition, lipoic acid significantly reduced the basal expression of NOX3, implicating ROS in this process. Other NADPH oxidase isoforms, such as Rac1, gp91 and p22, were also induced by cisplatin, with increases in expression being 478±85, 1000±102 and 8±1 fold, respectively (data not shown). The high fold induction of these latter transcripts over that observed for NOX3 might reflect their low basal expression in the cochlea as compared to NOX3.

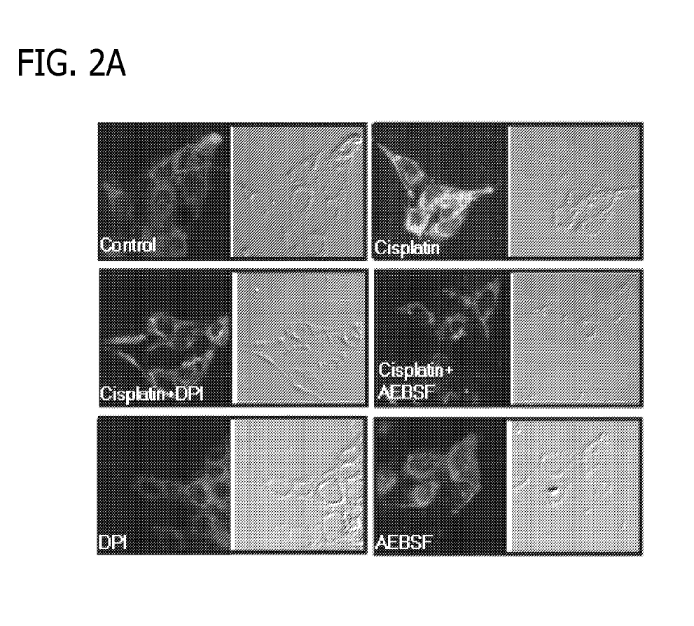

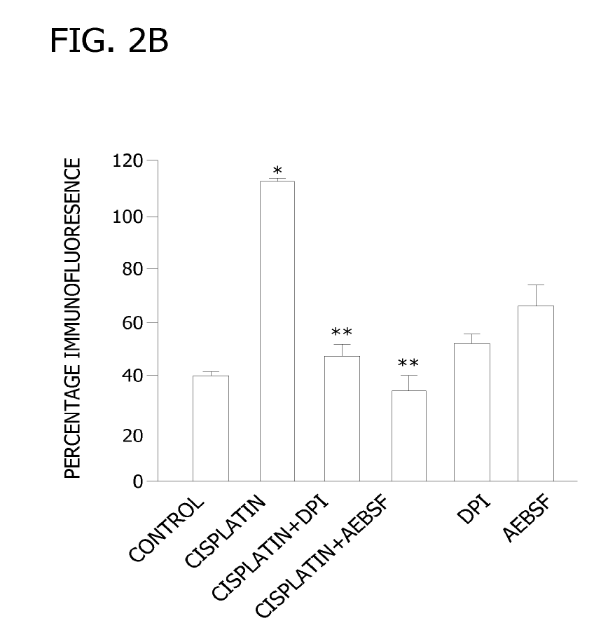

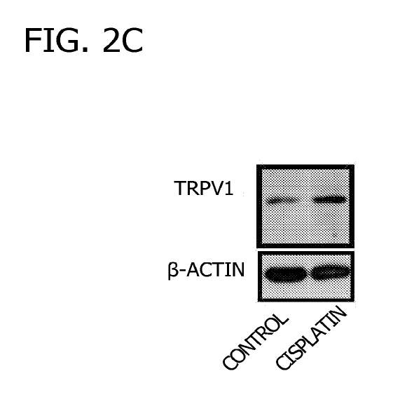

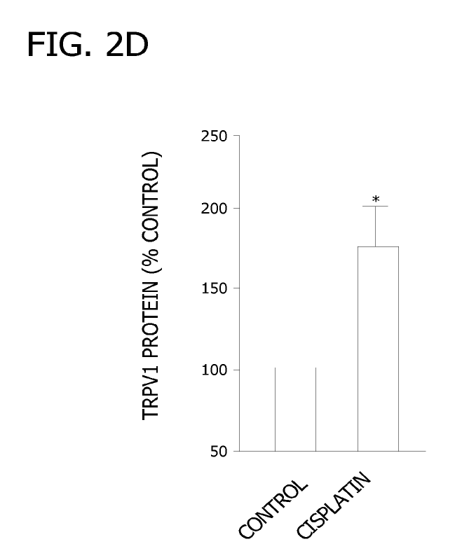

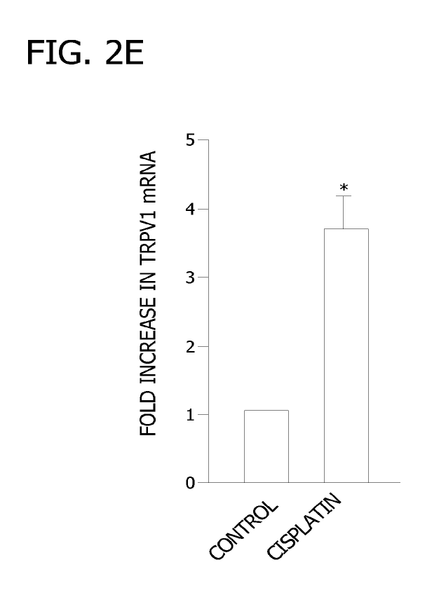

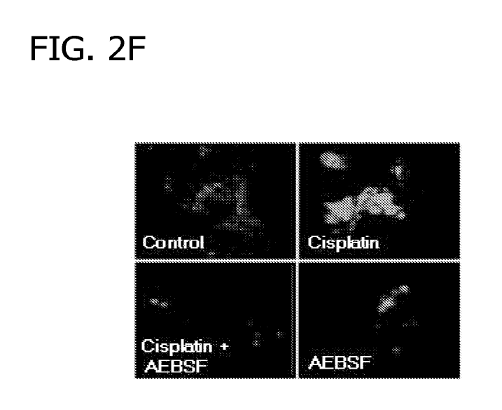

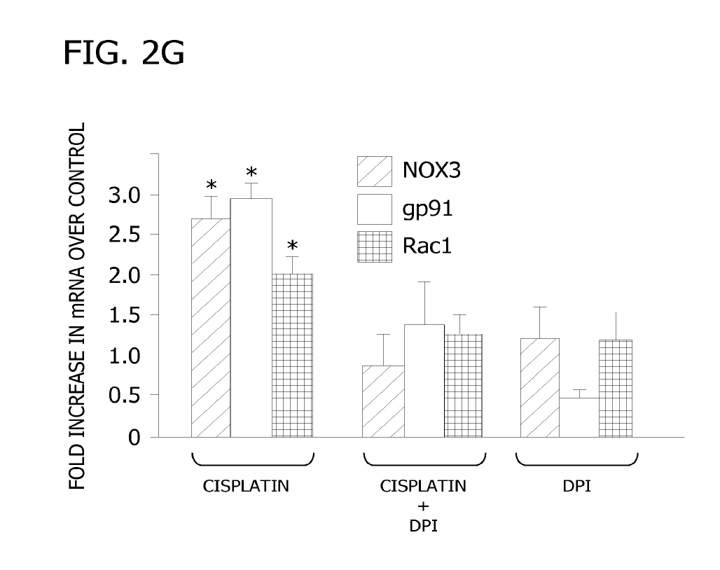

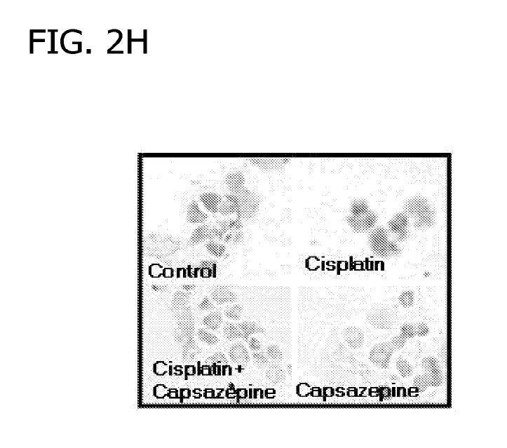

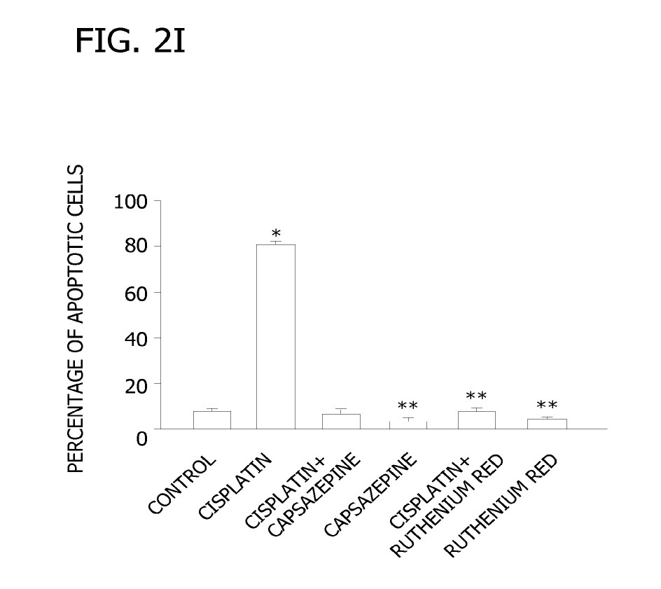

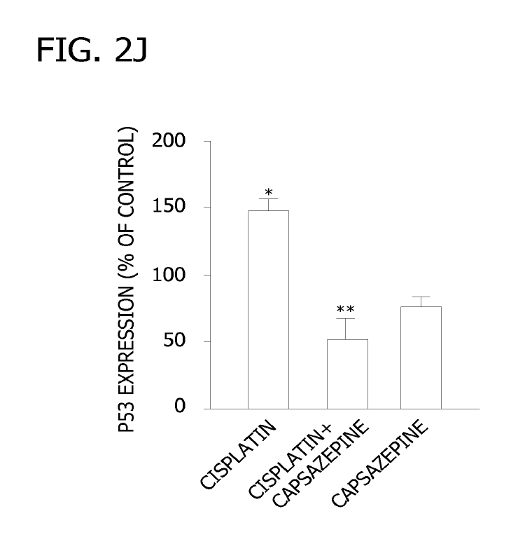

To further study the role of ROS generated via cochlear NADPH oxidases in the induction of TRPV1 in the cochlea, in vitro experiments were performed using the organ of Corti transformed hair cell line, UB/OC1 (Rivolta et al., Proc. R. Sco. Lond., 265, 1595-1603, 1998). These cells have been used previously to examine cisplatin induction of the kidney injury molecule (KIM-1) protein (Mukherjea et al., 2006, supra). Treatment of UB-OC1 cells with cisplatin (2.5 μM) for 24 h resulted in a 71±29% induction in TRPV1 immunoreactivity over control vehicle treated cells (FIG. 2b). Immunolabeling was quantitated by fluorescence imaging using a confocal microscope. Pretreatment of these cultures with either 100 μM AEBSF (Diatchuk et al., J. Biol. Chem., 272(20), 13292-13301, 1997) or 10 μM DPI (O'Donnell et al., Biochem J., 290, 41-49, 1993), inhibitors of NADPH oxidase, attenuated the increase observed with cisplatin, implicating ROS in this process (FIG. 2A). At these concentrations, these inhibitors did not significantly affect cell viability over a 24 h period. The immunofluorescence in these groups was reduced by 60 and 70%, respectively (FIG. 2B). The increase in TRPV1 protein expression produced by cisplatin in UB-OC1 cells was confirmed by Western blotting which showed a significant increase in TRPV1 protein (FIG. 2C) by 90%±10% (FIG. 2D). In addition, a significant increase in TRPV1 mRNA was observed in 24 h (FIG. 2E). UB-OC1 cells treated with cisplatin showed a robust increase in ROS generation, as determined by DCF2DA fluorescence. This increase was abolished in cells pretreated with AEBSF (100 μM), indicating of a role of NADPH oxidase activation (and possibly of NOX3) in the ROS generation induced by cisplatin (FIG. 2F). The increase in ROS generation was followed by a significant increase in mRNA encoding different NADPH oxidase subunits. A statistically significant increase in NOX3, gp91 and Rac1 by 2.7, 2.8 and 2.1 fold, respectively, was observed. The increases in expression of these genes were inhibited by DPI (10 μM), indicative of a role or NADPH oxidase activity in the induction of these subunits (FIG. 2G).

Treatment of UB-OC1 cells with a higher concentration of cisplatin (20 μM) for 24 h promoted apoptosis, as detected by TUNEL staining. At this concentration of cisplatin, ˜80% of apoptotic cells were obtained in UB-OC1 cultures. UB-OC1 cells pretreated for 30 min with either capsazepine or ruthenium red, inhibitors of TRPV1, and then administered cisplatin, showed significantly less apoptotic cells (˜7% of total cells) (FIGS. 2H,I). Neither capsazepine nor ruthenium red alone had any significant effect on cell apoptosis, compared to vehicle-treated control groups. These findings suggest that TRPV1 is an important intermediary for mediating cisplatin apoptosis in UB-OC1 cells. The increase in apoptosis by cisplatin was associated with increases in proapoptotic proteins such as p53 and Bax (FIGS. 2J,K). The increases in these proteins were attenuated by capsazepine (10 μM), implicating TRPV1 in this process. Cells pretreated with BAPTA-AM for 30 min prior the administration of cisplatin showed a substantial reduction in Bax protein, suggesting a role of intracellular Ca2+ in mediating apoptosis induced by cisplatin. Using Fura4-AM, it was shown that cisplatin increased intracellular Ca2+ in UB/OC-1 cells, which was inhibited using siRNA against TRPV1. This observation suggests that activation of TRPV1 by cisplatin results in intracellular Ca2+ accumulation, which can trigger apoptosis of UB/OC-1 cells.

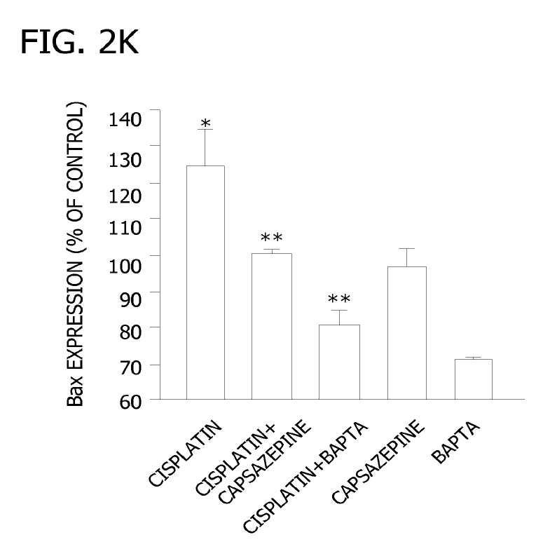

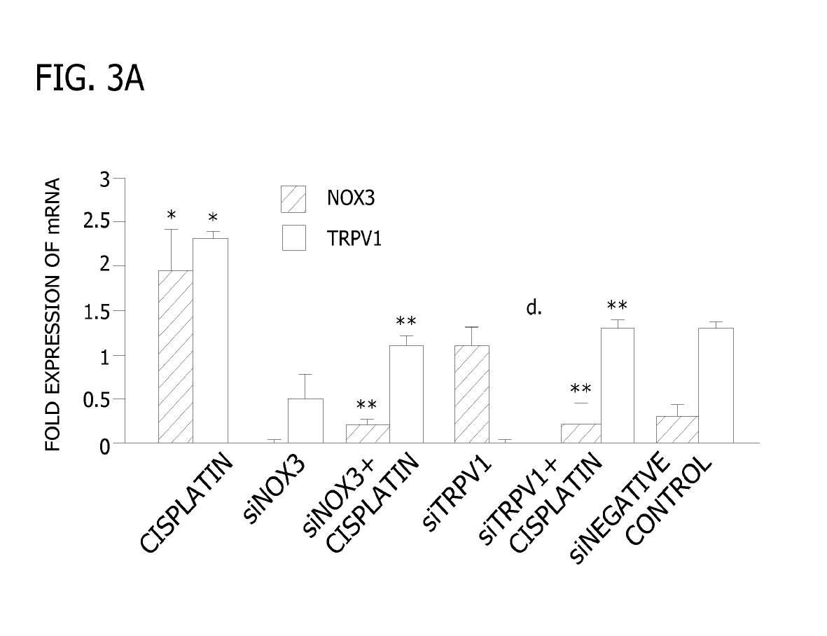

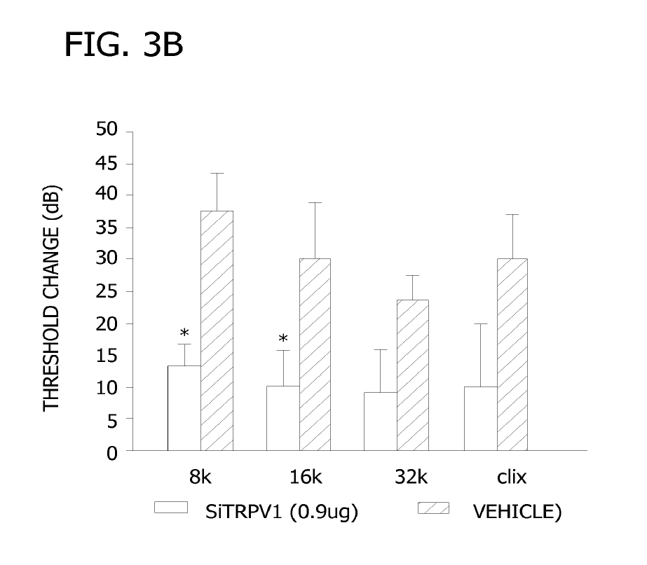

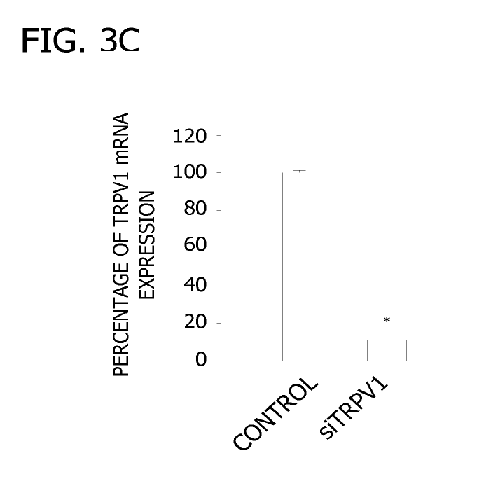

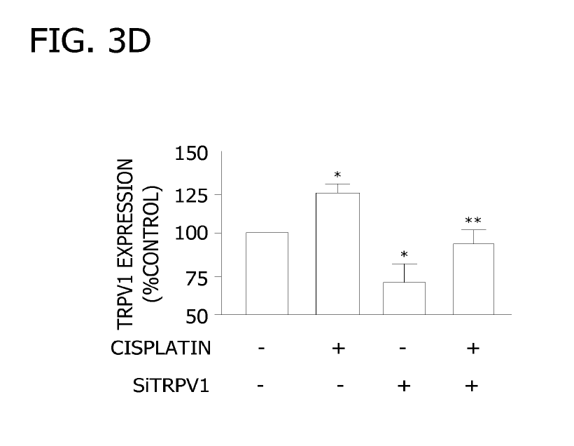

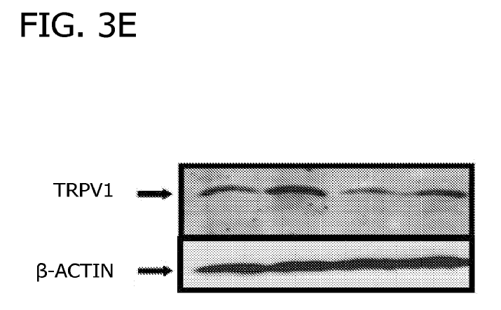

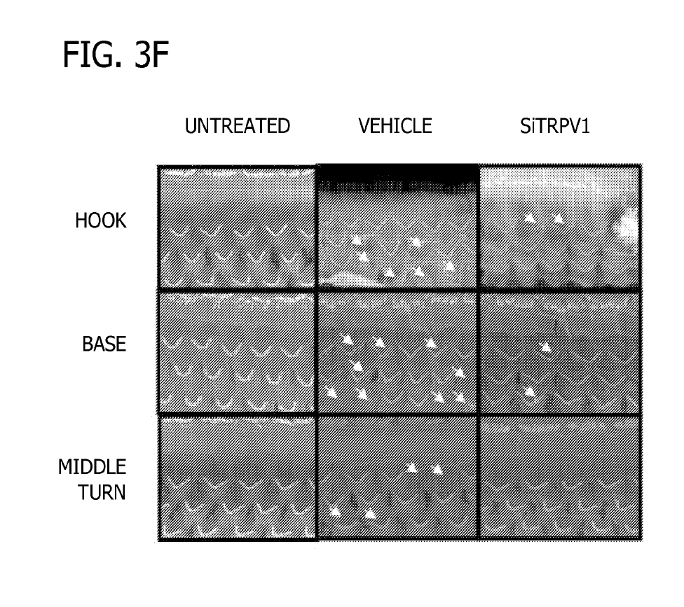

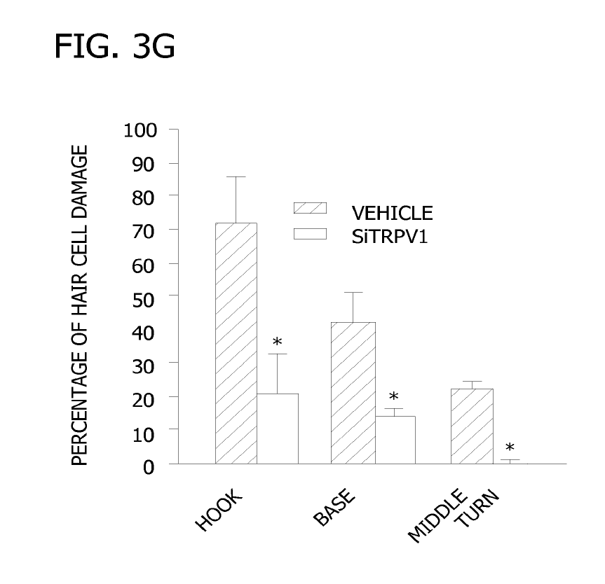

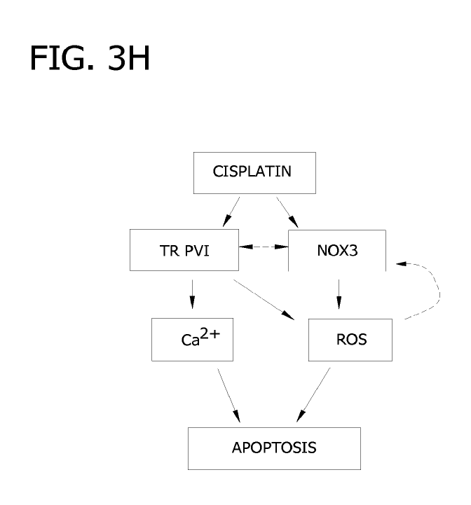

It was next determined whether selective inhibition of NOX3 synthesis by short interfering (si) RNA would reduce the expression of TRPV1 and possibly NOX3 itself. It was observed that NOX3 siRNA reduced the basal and cisplatin-stimulated expression of NOX3 by 24 h, without grossly affecting the morphology of the cells. Quantitation of TRPV1 mRNA by real time PCR indicated that NOX3 siRNA significantly reduced the basal and cisplatin-induced TRPV1 expression (FIG. 3A). Based on the previous data using inhibitors of NADPH oxidase (in vitro) and lipoic acid (in vivo), these data implicated NOX3 as a regulator of TRPV1 in UB-OC1 cultures. Interestingly, when cells were incubated with siRNA against TRPV1, attenuation of both the cisplatin-induced TRPV1 and NOX3 expression was observed, indicating possible co-regulation of these proteins (FIG. 3A).

To determine whether administration of TRPV1 siRNA would be effective against cisplatin-induced hearing loss, this agent was administered by round window application for 48 h and subsequently administered intraperitoneal cisplatin (13 mg/kg). Animals were tested 72 h later for auditory functions. Using cyanine-3 labeled siRNA, it was shown that round window application resulted in delivery of the fluorescent siRNA into the organ of Corti by 3 days after administration (the earliest time examined) and the signal persisted for ˜10 days. A significant increase in ABR thresholds was observed following cisplatin treatment alone which averaged 20-40 dB over an 8-32 kHz frequency range. However, in rats pre-treated with TRPV1 siRNA (0.9 μg/3 μl) 48 hrs prior to cisplatin administration, there was no significant shift in ABR threshold, implying effective protection against cisplatin ototoxicity (FIG. 3B). Significant protection against hearing loss was observed at 8 and 16 kHz tones, while a trend towards protection was observed using a 32 kHz tone (FIG. 3B). Real time PCR performed to determine the level of reduction in TRPV1 mRNA in the cochlea indicated an ˜85% decrease in expression following administration of a single concentration of TRPV1 siRNA and examining the cochlea on day 3 (FIG. 3C). In addition, a significant reduction of TRPV1 protein levels was observed in cochleas harvested from rats administered TRPV1 siRNA over the same time period (FIGS. 3D,E).

Morphological assessment of the outer hair cells by scanning electron microscopy indicated that cisplatin produced significant damage or loss of hair cells. Five rats were administered either vehicle or TRPV1 siRNA (3 μl) by round window application. The rats served as their own controls since one cochlea was treated with vehicle and the other with siRNA. Twenty four hours following the application of vehicle or siRNA, rats were infused with cisplatin (16 mg/kg, i.p) over a 30 min period and assessed for hair cell damage 3 days later. In 3 of the 5 rats treated with cisplatin, significant loss or damage of outer hair cells was observed in the cochlea pretreated with vehicle prior to cisplatin, but significant protection in the TRPV1 siRNA treated cochleas (FIG. 3F). The percentage of outer hair cell loss in the hook, base and middle turn of the cochlea in rats administered cisplatin (16 mg/kg, i.p.) was 72±14, 42±9 and 22±2.7 respectively (N=3). However, pretreatment with TRPV1 siRNA resulted in a significant reduction in percentage of hair cell loss to 21±12, 14±21 and no loss in the hook, base and the middle turns respectively (N=3) (statistically significant difference, p≦50.05) (FIG. 3G). One animal showed partial protection on the siRNA pretreated side, while no protection was observed in the cochlea of the remaining animal. No significant change in outer hair cell morphology was observed following the administration of TRPV1 siRNA alone (FIG. 3G).

### Noise

Prior to use, the animals were anesthetized and their heads were immobilized in a small animal stereotaxic with hollow earbars (David Kopf Instruments, Tujunga, Calif.). Animals were placed in a double-walled sound attenuating radio-frequency shielded booth (Industrial Acoustics Corporation) and their body temperatures were maintained at 37° C. using an animal warming blanket. Subdermal platinum alloy needle electrodes were attached with the active lead at the vertex and referred to a second electrode located over the temporal bone. The ground electrodes were placed over the neck muscles. Ten millisecond tone bursts (1, 2, 4, 8 and 16 kHz) were delivered monaurally through etymotic insert earphone placed directly into the ear bar. Auditory stimuli were presented at a rate of five per second with increasing intensity from 30 to 90 decibels (dB) sound pressure level (SPL) in 10 dB steps. The responses were amplified 100,000 times before being recorded. Auditory brainstem response (ABR) waveforms were repeated for each intensity. The response that clearly showed a reproducible wave form that displayed two or more peaks with a minimum amplitude of 0.5 μV was interpreted as the threshold response.

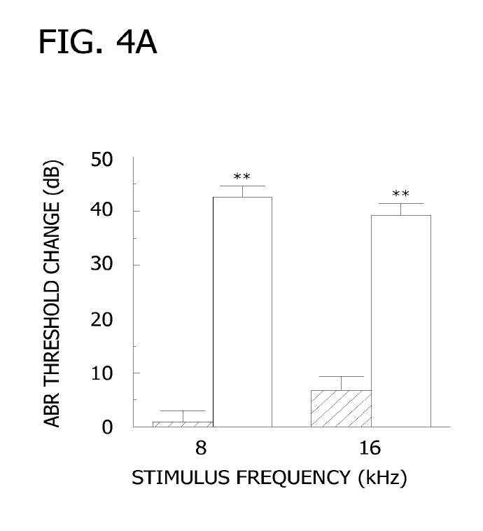

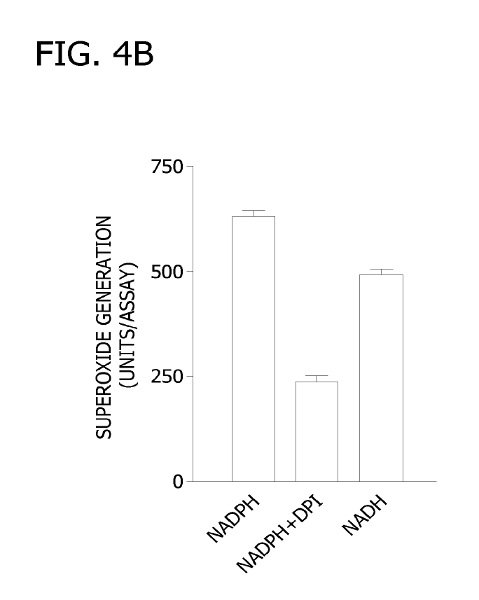

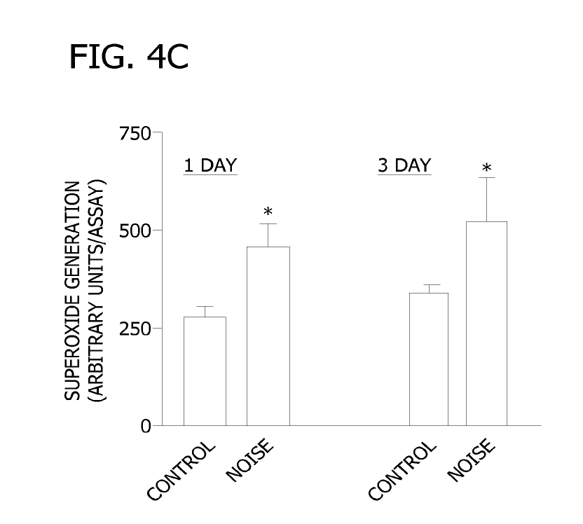

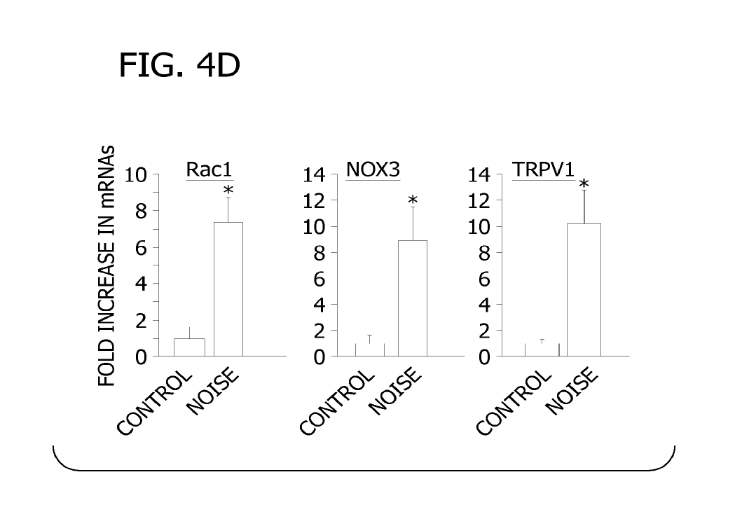

Noise exposure (90 dB, 6 h) produced a threshold shift in the ABR (between 8 and 16 kHz), which was observed when animals were tested after noise exposure. The shifts ranged between 38-42 dB at the two frequencies tested (FIG. 4A).

In order to determine whether noise exposure increased the activity of NADPH oxidase in the chinchilla cochlea, a major source of reactive oxygen species (ROS), the activity of NADPH oxidase in cytosolic preparations from the cochlea was examined. The activity of NAD(P)H oxidase was determined using 50 μg protein from cochlear extracts incubated with 100 μM β-NAD(P)H in a total volume of 200 μl of Hank's HEPES buffer (pH 7.4). The assay was initiated by the addition of 50 μM N,N′-dimethyl-9,9′-biacridinium dinitrate (Lucigenin) (Sigma, St. Louis, Mo.) to the incubation mixture. Samples were counted immediately using a table top luminometer (Berthold Detection Systems FB Luminometer, Zylux Corp., Maryville, Tenn.) with sampling time every 6 sec. Samples were counted over a period of 5 min and the fluorescence recorded over 2 min of stable readings were averaged for that samples. Samples were run in duplicate and the NAD(P)H oxidase activity was normalized to the protein concentrations in each sample.

Initial characterization of NADPH oxidase activity using N,N′-dimethyl-9,9′-biacridinium dinitrate (Lucigenin) as the fluorescent probe indicate blockade of enzyme activity by 10 μM diphenyleneiodonium chloride (Sigma, St. Louis, Mo.), a known inhibitor of this enzyme. Enzyme activity was greater using NADPH (100 μM) as a co-factor than with NADH (100 μM), indicative of NADPH oxidase (FIG. 4B).

Cochlear homogenates prepared from noise exposed animals (chinchilla were exposed to 96 dB noise for 6 h) demonstrated higher levels of NADPH oxidase activity, which were evident soon after the first noise exposure period (days 1) and on day 3 (i.e. 3 consecutive daily noise exposures). Cochlear NADPH oxidase activity increased from 278±27 (control ear) to 457±58 arbitrary units, following noise exposure for 1 day. Separate experiments were performed using different controls and 3 day noise exposed cochleae. These samples indicated that the NADPH oxidase activity increased from 338±23 (control ear) to 520±117 arbitrary units (FIG. 4C).

Additional experiments were performed in rats to determine whether the increase in NADPH oxidase activity observed with noise was associated with increased expression of NADPH oxidase subunits and TRPV1. Using primers for Rac1, NOX3 and TRPV1 in real time PCR assays, we observed statistically significant increases (˜8-10 fold) in the Rac1 and NOX3 subunits of NADPH oxidase and in TRPV1 (FIG. 4D).

### Aminoglycosides

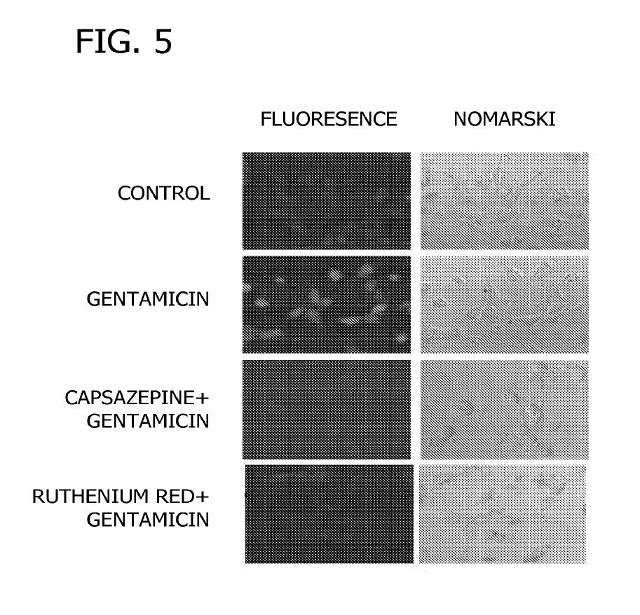

In this study, the mouse organ of Corti cell line (UB/OC-1) was used to examine the properties of aminoglycoside-induced ROS generation. For these experiments, cells were grown on coverslips to 60-80% confluency, then pretreated with vehicle (control and gentamicin groups), capsazepine (10 μM) or ruthenium red (20 μM) for 15 min. This was followed by vehicle (control) or gentamicin for 30 min. Cells were then loaded with 2′,7′-dichlorodihydrofluorescein diacetate (DCF2DA) and imaged by confocal microscopy. As shown in FIG. 5, the ROS generation of the gentamicin group was suppressed by capsazepine and ruthenium red (FIG. 5).

What is claimed is:

1. A method for preventing and/or reducing ototoxicity in a patient suffering from or at risk for developing ototoxicity caused by an ototoxic agent, noise, head or neck radiation, or tinnitus wherein the method comprises silencing TRPV1 in the patient.

2. The method of claim 1, wherein the silencing of the TRPV1 mRNA is achieved by administering siRNA directed against TRPV1 mRNA.

3. The method of claim 2, wherein a sense strand of the siRNA directed against TRPV1 mRNA comprises a nucleic acid sequence of SEQ ID NO: 1.

4. The method of claim 2, wherein a sense strand of the siRNA directed against TRPV1 mRNA comprises a sense nucleic acid sequence of SEQ ID NO: 2.

5. The method of claim 1, wherein the ototoxic agent is selected from the group consisting of an aminoglycoside and a platinum-containing chemotherapeutic agent.

6. The method of claim 5, wherein the aminoglycoside is selected from the group consisting of neomycin, paromomycin, ribostamycin, lividomycin, kanamycin, amikacin, tobramycin, viomycin, gentamicin, sisomicin, netilmicin, streptomycin, dibekacin, fortimicin, dihydrostreptomycin, and a combination thereof.

7. The method of claim 5, wherein the platinum-containing chemotherapeutic agent is selected from the group consisting of cisplatin and carboplatin.

8. The method of claim 7, wherein the platinum-containing chemotherapeutic agent is cisplatin.

9. The method of claim 2, wherein the siRNA directed against TRPV1 mRNA is administered locally.

10. The method of claim 9, wherein the siRNA directed against TRPV1 mRNA is administered to round window or intra-tympanically.

11. A method for preventing and/or reducing ototoxicity in a patient suffering from or at risk for developing ototoxicity caused by an ototoxic agent, noise, head or neck radiation, or tinnitus wherein the method comprises administering to the patient at least one siRNA selected from siRNAs directed against TRPV1 mRNA and NOX3 mRNA.

12. The method of claim 11, wherein the at least one siRNA directed against TRPV1 mRNA and at least one siRNA directed against NOX3 mRNA are administered to the patient.

13. The method of claim 11, wherein a sense strand of the siRNA directed against TRPV1 comprises a nucleic acid sequence selected from SEQ ID NO: 1 or SEQ ID NO: 2.

14. The method of claim 11, wherein a sense strand of the siRNA directed against NOX3 mRNA comprises SEQ ID NO: 3.

15. The method of claim 11, wherein the ototoxic agent is selected from the group consisting of an aminoglycoside and a platinum-containing chemotherapeutic agent.

16. The method of claim 15, wherein the aminoglycoside is selected from the group consisting of neomycin, paromomycin, ribostamycin, lividomycin, kanamycin, amikacin, tobramycin, viomycin, gentamicin, sisomicin, netilmicin, streptomycin, dibekacin, fortimicin, dihydrostreptomycin, and a combination thereof.

17. The method of claim 15, wherein the platinum-containing chemotherapeutic agent is selected from the group consisting of cisplatin and carboplatin.

18. The method of claim 17, wherein the platinum-containing chemotherapeutic agent is cisplatin.

19. The method of claim 11, wherein the at least one siRNA is administered locally.

20. The method of claim 19, wherein the at least one siRNA is administered to round window or intra-tympanically.

21. A method for preventing or reducing generation of reactive oxygen species in an inner ear of a patient, the method comprising administering to the patient at least one siRNA selected from the group consisting of a siRNA directed against TRPV1 mRNA and siRNA directed against NOX3 mRNA.

22. The method of claim 21, wherein a sense strand of the siRNA directed against TRPV1 mRNA comprises a nucleic acid sequence of SEQ ID NO: 1.

23. The method of claim 21, wherein a sense strand of the siRNA directed against TRPV1 mRNA comprises a nucleic acid sequence of SEQ ID NO: 2.

24. The method of claim 21, wherein a sense strand of the siRNA directed against NOX3 mRNA comprises a nucleic acid sequence of SEQ ID NO: 3.

25. The method of claim 21, wherein the generation of reactive oxygen species results from treatment of the patient with an ototoxic agent selected from the group consisting of an aminoglycoside and a platinum-containing chemotherapeutic agent.

26. The method of claim 25, wherein the aminoglycoside is selected from the group consisting of neomycin, paromomycin, ribostamycin, lividomycin, kanamycin, amikacin, tobramycin, viomycin, gentamicin, sisomicin, netilmicin, streptomycin, dibekacin, fortimicin, dihydrostreptomycin, and a combination thereof.

27. The method of claim 25, wherein the platinum-containing chemotherapeutic agent is selected from the group consisting of cisplatin and carboplatin.

28. The method of claim 27, wherein the platinum-containing chemotherapeutic agent is cisplatin.

29. The method of claim 21, wherein the at least one siRNA selected from siRNA directed against TRPV1 mRNA and NOX3 mRNA is administered locally.

30. The method of claim 29, wherein the at least one siRNA selected from siRNA directed against TRPV1 mRNA and NOX3 mRNA is administered to round window or intra-tympanically.
